(19) United States
(12) Patent Application Publication
(10) Pub. No.: US 2022/0211813 A1
(43) Pub. Date: Jul. 7, 2022

(54) METHODS FOR TREATMENT OF NIEMANN-PICK DISEASE TYPE C

(71) Applicant: LOMA LINDA UNIVERSITY, Loma Linda, CA (US)

(72) Inventors: Salvador SORIANO, Loma Linda, CA (US); Dong Kyu SHIN, Loma Linda, CA (US)

(73) Assignee: LOMA LINDA UNIVERSITY, Loma Linda, CA (US)

(21) Appl. No.: 17/594,167

(22) PCT Filed: Apr. 13, 2020
(86) PCT No.: PCT/US20/27931

§ 371 Date: Oct. 5, 2021

Related U.S. Application Data
(60) Provisional application No. 62/833,468, filed on Apr. 12, 2019.

(51) Int. Cl. A61K 38/18; A61K 31/137; A61K 31/445; A61K 45/06; A61P 3/06

(52) U.S. Cl. CPC A61K 38/1883; A61K 31/137; A61P 3/06; A61K 45/06; A61K 31/445

(57) ABSTRACT

Provided here are methods of treating Niemann-Pick disease type C (NPC) in a subject or delaying the onset of NPC in a subject by administering to the subject an immunomodulator, or a modulator of amyloid precursor protein (APP) function, or a combination thereof.

### CROSS-REFERENCE TO RELATED APPLICATION

This application claims the benefit of U.S. Provisional Application No. 62/833,468, filed Apr. 12, 2019.

### TECHNICAL FIELD

This disclosure generally relates to methods of treating or delaying the onset of Niemann-Pick disease type C (NPC) in a subject.

### BACKGROUND

Niemann-Pick disease type C (NPC) is a fatal neurodegenerative condition caused by genetic mutations of the NPC1 (Chr. 18q11.2) or NPC2 (Chr. 14q24.3) genes that encode the NPC1 and NPC2 proteins, respectively. Clinically, NPC1 and NPC2 dysfunctions result in an identical condition, with NPC1 mutations accounting for nearly 95% of the reported cases and NPC2 mutations only reported in a small number of families. Anatomically, the cerebellum is the most susceptible region to early neurodegeneration, marked by the progressive loss of cerebellar Purkinje neurons and early onset of cerebellar symptoms. Thus, the majority of the past and current research efforts are focused on elucidating the biological and pathological role of NPC1 in cerebellar degeneration.

NPC neurodegeneration is complex and incurable. To date, the precise functions of NPC1 and NPC2 remain incompletely understood, posing a challenge to understanding the pathogenesis and progression of NPC neurodegeneration. In humans and various models of NPC, previously characterized cellular dysfunctions of NPC include: endosomal lipid sequestration, neuroinflammation, dysregulated calcium signaling, mitochondrial dysfunction, increased oxidative stress, amyloid-beta (Δβ) aggregation, and tau-neurofibrillary tangles. While there are current lipid-targeting therapeutic efforts that are showing some clinical benefits, there is no FDA-approved therapy for NPC to date. The lipid dysregulation of NPC is perhaps the best-understood pathogenic mechanism and two lipid-targeting therapies are actively receiving attention, namely miglustat and beta-cyclodextrin. Miglustat is approved in western Europe for NPC and beta-cyclodextrin is under clinical trials for NPC in the United States. While these therapies appear to provide some clinical benefits, adjuvant therapies are still likely to be necessary, particularly considering the wide array of cellular dysfunctions of NPC.

### SUMMARY OF THE INVENTION

Embodiments of the disclosure include methods of treating or delaying onset of symptoms of Niemann-Pick disease type C in a subject. One such method includes administering to the subject an immunomodulator. The immunomodulator can be an immunosuppressor. The immunomodulator can be an inhibitor of Interferon I. The immunomodulator can be an inhibitor of Interferon II. The immunomodulator can be an inhibitor of interferon-gamma induced protein 10. The immunomodulator can be an inhibitor of a toll-like receptor. The immunomodulator can be an inhibitor of T-cell function. The immunomodulator can be Neuregulin 1. The immunomodulator can be an inhibitor of a fatty acid binding protein. The immunomodulator can be fingolimod.

Another method of treating or delaying onset of symptoms of Niemann-Pick disease type C in a subject includes administering to the subject a modulator of amyloid precursor protein (APP) function. The modulator of APP function can be a serotonin receptor agonist. The modulator of APP function can be donecopride. The modulator of APP function can be a specific 5-HT4 receptor agonist.

Another method of treating or delaying onset of symptoms of Niemann-Pick disease type C in a subject includes administering to the subject a combination of an immunomodulator and a modulator of APP function. The immunomodulator can be one or more therapies selected from: at least one interferon (IFN) inhibitor; at least one IP10/CXCL10 inhibitor; at least one CXCR3 inhibitor; at least one inhibitor of MIG/CXCL9, RANTES/CCL5, EOTAXIN/CCL11, and IL-10; at least one inhibitor of TLR; and at least one inhibitor of MCP1/CCL2, MIP-1a/CCL3, MIP-1β/CCL4, IL-1α, and KC/CXCL1. The modulator of APP function can be a serotonin receptor agonist. The modulator of APP function can be donecopride. The modulator of APP function can be a specific 5-HT4 receptor agonist.

Embodiments of the disclosure include methods of treating Niemann-Pick disease type C (NPC) in a subject. One such method includes administering to the subject one or more therapies selected from: at least one interferon (IFN) inhibitor; at least one IP10/CXCL10 inhibitor; at least one CXCR3 inhibitor; at least one immunosuppressive drug; an agent that prevents or reduces amyloid precursor protein (APP) loss of function; at least one inhibitor of MIG/CXCL9, RANTES/CCL5, EOTAXIN/CCL11, and/or IL-10; at least one inhibitor of TLR; and/or at least one inhibitor of MCP1/CCL2, MIP-1α/CCL3, MIP-1β/CCL4, IL-1α, and/or KC/CXCL1. In certain embodiments, the method reduces the neuroinflammation in the subject. In certain embodiments, the subject slows one or more symptoms of NPC. The symptoms can include one or more neurological symptoms, such as one or more of hypotonia, dystonia, hearing loss, balance disorder, ataxia, clumsiness, dysphagia, dysarthria, involuntary muscle contractions, seizure, insomnia, memory loss, and cognitive dysfunction.

Embodiments of the disclosure include methods of delaying the onset of Niemann-Pick disease type C (NPC) in a subject. One such method includes administering to the subject one or more therapies selected from: at least one interferon (IFN) inhibitor; at least one IP10/CXCL10 inhibitor; at least one CXCR3 inhibitor; at least one immunosuppressive drug; an agent that prevents or reduces amyloid precursor protein (APP) loss of function; at least one inhibitor selected from MIG/CXCL9, RANTES/CCL5, EOTAXIN/CCL11, and/or IL-10; at least one inhibitor of TLR; and at least one inhibitor selected from MCP1/CCL2, MIP-1α/CCL3, MIP-1β/CCL4, IL-1α, and KC/CXCL1. In certain embodiments, the therapy is administered to the subject when the subject has not shown any symptoms of NPC. In certain embodiments, the IFN inhibitor is an IFN-α inhibitor, an IFN-β inhibitor, or an IFN-γ inhibitor. The IP10/CXCL10 inhibitor can be methimazole or an anti-IP10/CXCL10 antibody. The CXCR3 inhibitor can be AMG487.

The immunosuppressive drug can be one or more of tacrolimus, mycophenolic acid, sirolimus, hydrocortisone, methylprednisolone, cyclosporin A, a nuclear factor-kB (NF-kB) inhibitor, a p38 mitogen-activated protein kinase (MAPK) inhibitor, a phosphatidylinositol 3-kinase (PI3K) inhibitor, a c-Jun NH2-terminal kinase (JNK) inhibitor, an extracellular signal-regulated kinase (ERK) inhibitor, a signal transducer and activator of transcription-1 (Stat1) inhibitor, elocalcitol, BXL-01-0029, or a T-cell receptor directed antibody. In certain embodiments, the agent that prevents or reduces APP loss of function can be a secreted domain of an APP protein or a nucleic acid encoding the same. In certain embodiments, the inhibitor of MIG/CXCL9, RANTES/CCL5, EOTAXIN/CCL11, and/or IL-10 is one or more of ketotifen, ibudilast, valproic acid, maraviroc, AG1478, or AG1478. The subject may have NPC1 or NPC2. The subject may have a mutation in the NPC1 gene or NPC2 gene.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. Embodiments are illustrated by way of example and not by way of limitation in the accompanying drawings.

FIGS. 1A-1L are graphical representations of the levels of interferon-gamma induced protein 10 (IP-10/CXCL10) (FIG. 1A), monokine induced by gamma interferon (MIG/CXCL9) (FIG. 1B), monocyte chemoattractant protein-1 (MCP-1/CCL2) (FIG. 1C), macrophage inflammatory protein-1-alpha (MIP-1α/CCL3) (FIG. 1D), macrophage inflammatory protein-1-beta (MIP-1(3/CCL4) (FIG. 1E), regulated on activation normal T cell expressed and secreted (RANTES/CCL5) (FIG. 1F), macrophage colony-stimulating factor (M-CSF) (FIG. 1G), interleukin-1-alpha (IL-1α) (FIG. 1H), keratinocyte chemoattractant (KC/CXCL1) (FIG. 1I), Interleukin-15 (IL-15) (FIG. 1J), eotaxin (CCL11) (FIG. 1K) and leukemia inhibitory factor (LIF) (FIG. 1L) in the cerebella of wild-type and Npc1^{-/-} mice at 3 and 12 weeks of age.

FIGS. 2A-2N are graphical representations of the levels of interleukin-1-beta (IL-1β) (FIG. 2A), interleukin-2 (IL-2) (FIG. 2B), interleukin-4 (IL-4) (FIG. 2C), interleukin-7 (IL-7) (FIG. 2D), interleukin-17 (IL-17) (FIG. 2E), granulocyte colony-stimulating factor (G-CSF) (FIG. 2F), interferon-gamma (IFN-γ) (FIG. 2G), interleukin-5 (IL-5) (FIG. 2H), interleukin-6 (IL-6) (FIG. 21), interleukin-9 (IL-9) (FIG. 2J), interleukin-10 (IL-10) (FIG. 2K), interleukin-12 (IL-12) (p40) (FIG. 2L), macrophage inflammatory protein-2 (MIP-2/CXCL2) (FIG. 2M), and vascular endothelial growth factor (VEGF) (FIG. 2N) in the cerebella of wild-type and Npc1^{-/-} mice at 3 and 12 weeks of age.

FIGS. 3A and 3B show the expression of genes in the Npc1^{-/-} cerebellar transcriptome utilizing the Gene-Set Enrichment Analysis (GSEA).

FIG. 4A shows the mapping of the molecular functions and relationships of differentially expressed interferon-responsive genes identified within the Npc1^{-/-} cerebellar transcriptome using the Ingenuity Pathway Analysis software (IPA, Qiagen). Red indicates upregulation and green indicates downregulation. DEGs plotted in their respective sub-cellular location; p<0.05 with each FC-value listed below the gene symbol. *Duplicate identifiers used for the same gene. FIG. 4B presents the IPA key for molecule shape, color, and interaction.

FIG. 5 shows the mapping of nine IFN-γ-responsive genes: Lgals3, Mcp1/Ccl2, Lcn2, Itga5, IP10/Cxcl10, Tlr4, Tgfb1, Casp1, and Rantes/Ccl5 that are directly related to the activation of microglia. Red indicates upregulation and green indicates downregulation.

FIG. 6 shows the merged network of IFN-γ- and IFN-α-responsive DEGs involved in microglial activation, anti-viral response, activation of T-lymphocytes, and chemotaxis of T-lymphocytes.

FIG. 7 shows the mapping of genes downstream of activated toll-like receptor (TLR) in pre-symptomatic Npc1^{-/-} mouse cerebella. Red indicates upregulation and green indicates downregulation.

FIG. 8 is a schematic representation of the mechanism of NPC neuroinflammation.

FIG. 9 is a GSEA that reveals the activation of Interferon Gamma Response gene sets in Npc1^{-/-}/App^{-/-} mouse cerebella compared with the three remaining genotypes (Npc1−/−/App−/− vs. remaining genotypes). ES=enrichment score, NES=normalized enrichment score, FDR-q=false discovery rate q-value.

FIG. 10A shows the mapping of genes involved in IFN-γ downstream signaling in the Npc1^{-/-}/App^{-/-} cerebellar transcriptome. Red indicates upregulation and green indicates downregulation. FIG. 10B presents the IPA key for molecule shape, color, and interaction.

FIG. 11 is a GSEA that reveals the activation of Interferon Alpha Response gene sets in Npc1^{-/-}/App^{-/-} mouse cerebella compared with the three remaining genotypes (Npc1^{-/-}/App^{-/-} vs. remaining genotypes). ES=enrichment score, NES=normalized enrichment score, FDR-q=false discovery rate q-value.

FIG. 12 shows mapping of the IFN-α-responsive genes in the Npc1^{-/-}/App^{-/-} mouse cerebella as compared with age-matched wild-type littermates (Npc1^{+/+}/App^{+/+}). All plotted DEGs meet the significance cutoff of fold-change (absolute FC>1.5) and p-value (p<0.05). *Duplicate identifiers used for the same gene. A detailed key for IPA molecular shape, color, and interaction is provided in FIG. 10B.

FIGS. 13A-13E are graphical representations demonstrating that the progressive loss of functional App allele in NPC mouse model (Npc1^{-/-}/App^{+/-} and Npc1^{-/-}/App^{-/-}) resulted in significant increase of pro-inflammatory cytokines at 3 weeks of age. FIG. 13A is a graphical representation of IP-10/CXCL10 expression in Npc1^{-/-}/App^{+/+} in the pre-symptomatic mouse cerebella. FIGS. 13B-13D are graphical representations of the expression of RANTES/CCL5, EOTAXIN/CCL11, and IL-10, respectively, that were also significantly increased in Npc1^{-/-}/App^{+/-} and/or Npc1^{-/-}/App^{-/-} mouse cerebella compared with wild-type (Npc1^{+/+}/App^{+/+}) and/or Npc1^{-/-}/App^{+/+}. FIG. 13E is a graphical representation of the expression of IL-1β expression in Npc1^{-/-}/App^{+/-} and/or Npc1^{-/-}/App^{-/-} mouse cerebella compared with wild-type (Npc1^{+/+}/App^{+/+}) and/or Npc1^{-/-}/App^{+/+}. Values are means±SEM. *p<0.05, **p<0.01. *=compared to Npc1^{+/+}/App^{+/+}; \hat{ }=compared with Npc1^{+/+}/App^{-/-}; #=compared with Npc1^{-/-}/App^{+/+}.

FIGS. 14A-14O are immunohistochemically stained-images to examine the filtration of CD3+ T cells in cerebellum. Shown for comparison as a positive control is CD3 staining of T cells in mice following a traumatic brain injury protocol: FIGS. 14A-14C—Npc1^{+/+}/App^{+/+} mice cerebella at 12 weeks of age, stained from DAPI, CD3 and DAPI+CD3, respectively; FIGS. 14D-14F—Npc1^{-/-}/App^{+/+} mice at terminal disease stage, stained from DAPI, CD3 and DAPI+CD3, respectively; FIGS. 14G-14I—Npc1^{+/+}/App^{-/-} mice at 12 weeks of age, stained from DAPI, CD3 and DAPI+CD3, respectively; FIGS. 14J-14L—App^{-/-}/Npc1^{-/-} mice at terminal disease stage, stained from DAPI, CD3 and DAPI+CD3, respectively; FIGS. 14M-14O—Traumatic brain injury positive control, stained from DAPI, CD3 and DAPI+CD3, respectively. Shown is the lesion area. g: granular layer of the cerebellum; m: molecular layer of the cerebellum. White asterisks show CD3+ cells and white arrows show areas of stained patterns that are artifacts, as they appear in all genotypes and all ages tested.

FIG. 15 shows the mapping of genes involved in the exacerbation of microglial activation pathway in Npc1^{-/-}/App^{-/-} mouse cerebella.

FIG. 16 shows the mapping of genes involved in the exacerbation of the antiviral response in Npc1^{-/-}/App^{-/-} mouse cerebella.

FIG. 17 shows the mapping of genes involved in the exacerbation of the antimicrobial response in Npc1^{-/-}/App^{-/-} mouse cerebella.

FIG. 18 shows the mapping of genes involved in the exacerbation of T-lymphocyte pathway in Npc1^{-/-}/App^{-/-} mouse cerebella.

FIG. 19 shows the mapping of genes involved in the exacerbation of activation of T-lymphocyte co-stimulatory receptor CD28 in Npc1^{-/-}/App^{-/-} mouse cerebella.

FIG. 20 shows the mapping of genes involved in the exacerbation of chemotaxis of T-lymphocytes pathway in Npc1^{-/-}/App^{-/-} mouse cerebella.

FIG. 21 shows the mapping of genes involved in the exacerbation of antigen presentation pathway in Npc1^{-/-}/App^{-/-} mouse cerebella.

FIG. 22 shows the mapping of genes involved in the activation of dendritic cells in the Npc1−/−/App−/− mouse cerebella as a result of APP loss of function.

FIG. 23 shows the mapping of genes involved in the activation of APC-associated co-stimulatory molecules in Npc1^{-/-}/App^{-/-} mouse cerebella.

FIGS. 24A-24N are graphical representation of the pleotropic and variable cytokine/chemokine expressions in the terminal stage cerebella of Npc1−/−/App+/+, Npc1−/−/App+/−, and Npc1−/−/App−/− compared with Npc1+/+/App+/+ and Npc1+/+/App−/−. Values are means±SEM. *p<0.05, **p<0.01. *=compared with Npc1+/+/App+/+; \hat{ }=compared with Npc1+/+/App−/−; #=compared with Npc1−/−/App+/+.

FIG. 25A-25L are immunohistochemically stained-images to examine the infiltration of CD3+ T cells in cerebellum. FIGS. 25A-25C are images of Npc1^{+/+}/App^{+/+} mice cerebella. FIGS. 25D-25F are images of Npc1^{-/-}/App^{+/+} mice cerebella. FIGS. 25G-25I are images of Npc1^{-/-}/App^{-/-} mice cerebella. FIGS. 25J-25L are images of App^{-/-}/Npc1^{-/-} mice cerebella.

### DETAILED DESCRIPTION OF THE INVENTION

Niemann-Pick disease type C1 (NPC) is a fatal neuro-visceral condition caused by the genetic mutations in the NPC1 or NPC2 gene. Historically, NPC is considered as a lysosomal storage disease due to the significant accumulation of various lipids (cholesterol, sphingosine, sphingolipids, glycolipids, glycosphingolipids) in the endo-lysosomes of genetically affected cells. The disclosure provides a genome-wide transcriptome study that identifies a co-activation of interferon-γ (IFN-γ) and IFN-α downstream signaling pathway that is activated in pre-symptomatic NPC. The genome-wide transcriptome study characterized the following immune response pathways to be activated in pre-symptomatic NPC that are direct targets for therapy: microglial activation, anti-viral response, T-lymphocyte activation, chemotaxis of T-lymphocytes, and antigen presentation. There was a significantly increased protein level of IP-10/CXCL10, a downstream effector of IFN-γ and IFN-α pathways, in pre-symptomatic NPC.

Embodiments of the disclosure include methods of treating NPC in a subject by administering one or more immunosuppressors. In an embodiment, the immunosuppressor is an inhibitor of Interferon I. In an embodiment, the immunosuppressor is an inhibitor of Interferon II. In an embodiment, the immunosuppressor is an inhibitor of IP10/CXCL10 signaling. In an embodiment, the immunosuppressor is an inhibitor of CXCR3. Embodiments of the disclosure include methods of treating NPC in a subject by administering one or more TLR inhibitors. Embodiments of the disclosure include methods of treating NPC in a subject by administering one or more immunosuppressors of T-cell function.

Embodiments of the disclosure include methods of treating NPC in a subject by administering one or more immunomodulators. In an embodiment, the immunomodulator is Neuregulin 1. Administration of Neuregulin 1 can minimize inflammation-induced damage in the NPC brain by minimizing the IP10/CXCL10 dysregulation is present in the early stages of the disease. In an embodiment, the immunomodulator is a FABP inhibitor. Administration of one or more FABP inhibitors can reduce the damage of microglial activation, which is also prominent in the early NPC brain. In an embodiment, the immunomodulator is fingolimod, a sphingosine-1-phosphate receptor regulator. Administration of fingolimod can neutralize the negative impact of TLR4 and IP10 on early NPC.

Embodiments of the disclosure include methods of treating NPC in a subject by administering one or more modulators of amyloid precursor protein (APP) function. In an embodiment, the modulator of APP function is a serotonin receptor agonist. In an embodiment, a serotonin receptor agonist is donecopride. In an embodiment, the modulator of APP function is a specific 5-HT4 receptor agonist, such as RS67333. Embodiments of the disclosure include methods of treating NPC in a subject by administering an enzyme inhibitor to reduce cholesterol oxidation.

Embodiments of the disclosure include methods of treating a subject who has a NPC diagnosis. The subject can be diagnosed by blood-based testing for biomarkers (oxysterols, lysosphingolipids, bile acid metabolites). The subject can be diagnosed by gene sequencing of NPC1 and NPC2 genes, or fragments thereof. The subject can be diagnosed by filipin staining or by cholesterol esterification test.

Embodiments of the disclosure include methods of treating NPC in a subject by administering to the subject one or more therapies selected from: at least one interferon (IFN) inhibitor; at least one interferon-gamma induced protein 10 (IP10/CXCL10) inhibitor; at least one CXCR3 inhibitor; at least one immunosuppressive drug; an agent that prevents or reduces amyloid precursor protein (APP) loss of function; at least one inhibitor of MIG/CXCL9, RANTES/CCL5, EOTAXIN/CCL11, and/or IL-10; at least one inhibitor of TLR; and/or at least one inhibitor of MCP1/CCL2, MIP-1α/CCL3, MIP-1β/CCL4, IL-1α, and/or KC/CXCL1. In certain embodiments, the methods reduce the neuroinflammation in the subject. In certain embodiments, the subject slows one or more symptoms of NPC. The symptoms can include one or more neurological symptoms, such as one or more of hypotonia, dystonia, hearing loss, balance disorder, ataxia, clumsiness, dysphagia, dysarthria, involuntary muscle contractions, seizure, insomnia, memory loss, and cognitive dysfunction.

As used herein, the term “Niemann-Pick disease type C (NPC)” refers to a neuro-visceral disease associated with mutations in the NPC1 gene or NPC2 gene.

As used herein, the term “inhibitor” refers to any agent or molecule (e.g., organic small molecules, biologics, drugs, antibodies, peptides, proteins, and the like) that inhibits or reduces the expression, amount, and/or biological effect of a target protein or oligonucleotide, either directly or indirectly. For example, an inhibitor can be an antibody that specifically binds to IP10/CXCL10. In another example, an inhibitor can indirectly inhibit or reduce the biological effect of IP10/CXCL10 by binding to CXCR3, which interacts with IP10/CXCL10.

As used herein, the term “treat,” “treating,” or “treatment” generally means obtaining a desired pharmacologic and/or physiologic effect. It may refer to any indicia of success in the treatment or amelioration of a disease (e.g., NPC), including any objective or subjective parameter such as abatement, remission, improvement in patient survival, increase in survival time or rate, diminishing of symptoms or making the disease more tolerable to the patient, slowing in the rate of degeneration or decline, or improving a patient's physical or mental well-being. The effect of treatment can be compared to an individual or pool of individuals not receiving the treatment, or to the same patient prior to treatment, or at a different time during treatment.

As used herein, the term “administer,” “administering,” or variants thereof means introducing a therapeutically effective dose of a compound disclosed herein into the body of a patient in need of it to treat or delay onset of symptoms of NPC.

As disclosed herein, one or more IFN inhibitors (e.g., Type I IFN inhibitors and Type II IFN inhibitors) can be used to treat NPC in a subject or delay the onset of NPC in a subject. A comparative inflammatory cytokine analysis in both pre-symptomatic (3-week) and terminal stage (11 to 12-week) cerebella of Npc1^{-/-} mice (BALB/cNctr-Npc1^{miN}/J) was conducted in order to identify the early and late inflammatory markers of NPC neurodegenerative cascade. In both the early and terminal stage Npc1^{-/-} mouse cerebella, interferon-gamma (IFN-γ) responsive cytokines were significantly elevated. Particularly, interferon-gamma induced protein 10 (IP10/CXCL10) is significantly upregulated in the pre-symptomatic stage and further exacerbated in the terminal stage Npc1^{-/-} cerebella. Transcriptome analysis of the pre-symptomatic cerebella confirmed the activation of IFN-γ downstream genes and IFN-α downstream genes.

Embodiments include methods of treating NPC in a subject by administering to the subject one or more Type I IFN inhibitors that can be IFN-α inhibitors or IFN-β inhibitors. Examples of IFN-α inhibitors and IFN-β inhibitors are available in the art, e.g., the inhibitors described in Gage et al. 2016, which provides compounds from the Small Diversity Set Compound Library (Dundee Drug Discovery Unit, University of Dundee, UK). For example, StA-IFN-1 (4-(1-acetyl-1H-indol-3-yl)-5-methyl-2,4-dihydro-3H-pyrazol-3-one) and StA-IFN-4 (2-[(4,5-dichloro-6-oxo-1(6H)-pyridazinyl) methyl]-8-methyl-4H-pyrido[1,2-a]pyrimidin-4-one) are compounds in the library and can be used to treat NPC in a subject or delay the onset of NPC in a subject. An example of an IFN-β inhibitor is BX795, as described in Clark et al. J Biol Chem, 284(21):14136-46, 2009, which blocks the phosphorylation, nuclear translocation, and transcriptional activity of interferon regulatory factor 3 and, hence, the production of IFN-β. Another example of an IFN-β inhibitor is ruxolitinib, which is a JAK1/2 inhibitor that reduces IFN-β toxicity. Examples of IFN-α inhibitors include, but are not limited to, bortezomib, ONX 0914, and carfilzomib. These inhibitors reduce IFN-α production in vitro and in vivo as shown in a murine lupus model.

Embodiments include methods of treating NPC in a subject by administering to the subject one or more Type II IFN inhibitors that include IFN-γ inhibitors. IFN-γ is a master regulator of the adaptive immune activation that is crucial in the transition from the innate immune response to the antigen-specific adaptive immune response. Therefore, the significant expression of IFN-γ responsive IP-10/CXCL10 in 3-week old Npc1^{-/-} cerebella indicates that IFN-γ downstream signaling may be activated early in the neurodegenerative cascade of NPC. An example of an IFN-γ inhibitor is TPCA-1, as described in Pododlin et al. J Pharmacol Exp Ther. 312(1):373-81, 2005, which is an IKK-2 inhibitor that blocks IFN-γ by about 50%. Examples of IFN-γ inhibitors also include anti-IFN-γ antibodies, e.g., as described in Grau et al. 1989.

Embodiments include methods of treating NPC in a subject by administering to the subject one or more inhibitors to IFNs, which include, but are not limited to, inhibitors of IFN-β (such as, cardiac glycosides, including bufalin), monoclonal antibodies against IFN-γ (such as, clones GZ4, 1-D1K, MT126L, 45F, 30S, 111W, 42H, 40K, 7-B6-1, 124i, 124i, G23, and 11i, as described in Olex et al. 2016), monoclonal antibody against type I interferon receptor (such as, anifrolumab that blocks the activity of IFN-α and IFN-β), monoclonal antibody against IFN-α (such as, sifalimumab), and monoclonal antibody against IFN-γ (such as, emapalumab (Gamifant)).

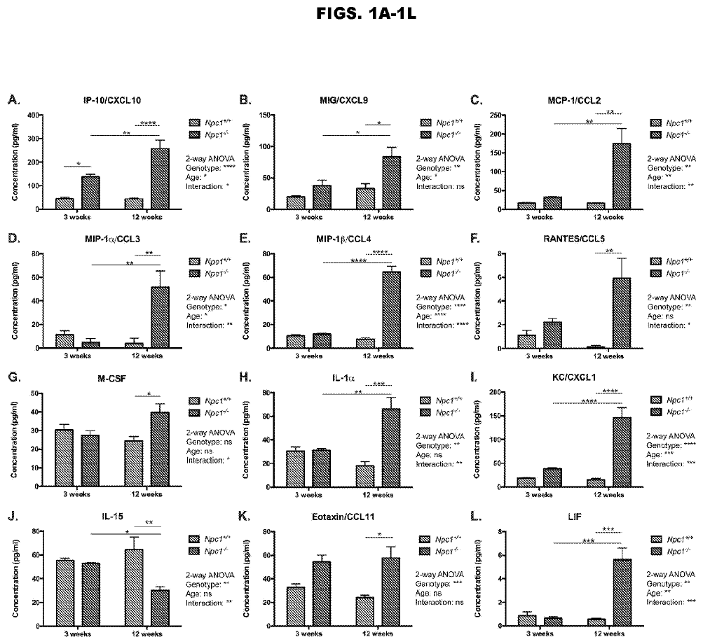

In an embodiment, one or more IP10/CXCL10 inhibitors can be used to treat NPC in a subject or delay the onset of NPC in a subject. IP10/CXCL10 was the only molecule significantly elevated in the Npc1^{-/-} cerebella at the early stage of three weeks, compared with the cerebella of the wild type control littermates (FIG. 1A). IP-10/CXCL10 levels also remained significantly increased in the terminal stage Npc1^{-/-} cerebella, compared with age-matched wild-type (Npc1^{+/+}) littermates (FIG. 1A). Further, IP10/CXCL10 is also a potent downstream effector of IFN-γ. Thus, the early elevated level of IP10/CXCL10 indicates that it contributes to the subsequent neuroinflammation and neurodegenerative cascade of NPC.

In some embodiments, an IP10/CXCL10 inhibitor directly targets IP10/CXCL10. Examples of IP10/CXCL10 inhibitors that directly inhibit the IP10/CXCL10 include, but are not limited to, anti-IP10/CXCL10 antibodies. Examples of anti-IP10/CXL10 antibodies include, but are not limited to, those described in U.S. Patent Publication No. US 2010/0021463 which is incorporated by reference herein in its entirety, those described in Australian Patent Publication No. AU2004298492B2 which is incorporated by reference herein in its entirety, NI-0801 (Novimmune), eldelumab, 1B6 (as described in Bonvin et al. 2017), 1F11 (as described in Khan et al. 2000), 1A4 (as described in Bonvin et al. 2017 and Bonvin et al. 2015).

Embodiments include methods of treating NPC in a subject by administering an IP10/CXCL10 inhibitor, such as methimazole that reduces or inhibits IP10/CXCL10 secretion. Other examples of an IP10/CXCL10 inhibitor are molecules that can act as antagonists of IP10/CXCL10, e.g., the truncated IP10/CXCL10 molecules. Another example of an IP10/CXCL10 inhibitor is DT390-IP-10, which consists of IP-10 (a ligand of CXCR3) as the targeting moiety and a truncated diphtheria toxin (DT390) as the toxic moiety. Another example of an IP10/CXCL10 inhibitor is a CXCL10 DNA vaccine, which induces the production of anti-CXCL10 Ab in vivo. Another example of an IP10/CXCL10 inhibitor is Rp-8-Br-cAMP, which blocks IP10/CXCL10 mediated inhibition of VEGF-mediated angiogenesis.

Embodiments include methods of treating NPC or delaying the onset of NPC in a subject by administering the microRNA miR-21. Embodiments include methods of treating NPC or delaying the onset of NPC in a subject by administering atorvastatin to decrease IP10/CXCL10.

Embodiments include methods of treating NPC or delaying the onset of NPC in a subject by administering one or more CXCR3 inhibitors. As IP-10/CXCL10 levels are significantly increased in the early and terminal stages Npc1^{-/-} cerebella and IP-10/CXCL10 binds to CXCR3 receptor on natural killer (NK) cells and various subtypes of lymphocytes to promote pathology, one or more CXCR3 inhibitors can be used to inhibit the binding of IP-10/CXCL10 to CXCR3. Embodiments include methods of treating NPC or delaying the onset of NPC in a subject by administering AMG487, an 8-azaquinazolinone that is a CXCR3 inhibitor.

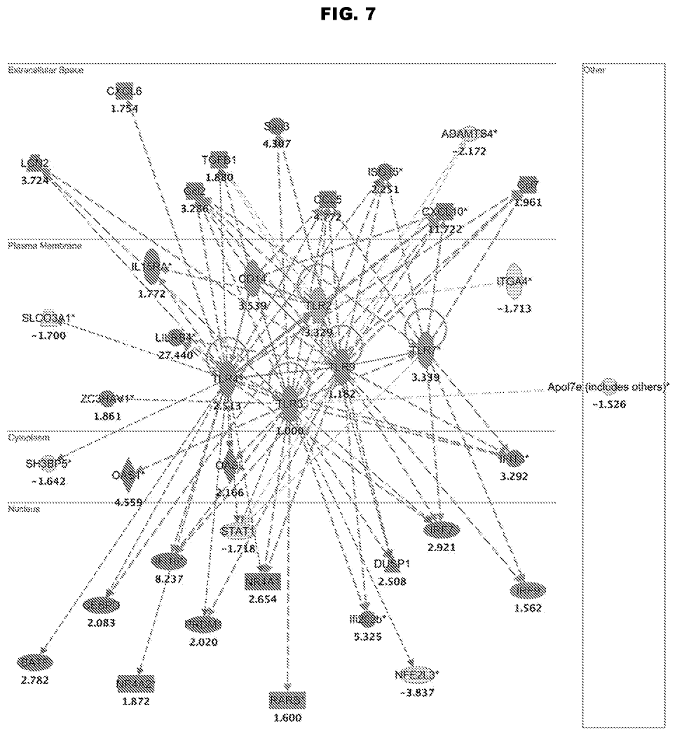

Genome-wide transcriptome analysis of pre-symptomatic NPC (Npc1^{-/-}) mouse cerebella highlighted activation of genes downstream of toll-like receptor (TLRs) signaling (FIG. 7). Both plasma membrane-bound TLRs (TLR2 and TLR4) that recognize microbial membrane material (e.g., LPS), as well as endosomal-membrane bound TLRs (TLR3, TLR7, and TLR9) that recognize microbial nucleic acids are implicated. Additionally, TLR4 co-receptor CD14, as well as TLR associated proteins MD-2 and MyD88, are also implicated. Embodiments include methods of treating NPC or delaying the onset of NPC in a subject by administering one or more of a TLR inhibitor, a CD14 inhibitor, a MD-2 inhibitor, or MyD88 (myeloid differentiation primary response protein) inhibitors.

Embodiments include methods of treating NPC or delaying the onset of NPC in a subject by administering a TLR inhibitor that is a small molecule. Examples of TLR inhibitors include, but are not limited to, VB-201 (a small molecule inhibitor of TLR2), TAK-242 (resatorvid) (a small molecule inhibitor of TLR4), fluvastatin (a small molecule inhibitor of TLR4), simvastatin (a small molecule inhibitor of TLR4), atorvastatin (a small molecule inhibitor of TLR4), candesartan (a small molecule inhibitor of TLR2/4), valsartan (a small molecule inhibitor of TLR2/4), chloroquine (a small molecule inhibitor of TLR3), chloroquine (a small molecule inhibitor of TLR7/8/9), hydroxychloroquine (a small molecule inhibitor of TLR7/8/9), CpG-52364 (a small molecule inhibitor of TLR7/8/9), and SM934 (a small molecule inhibitor of TLR7/9). Examples of MyD88 inhibitors include, but are not limited to, ST2825. Examples of CD14 inhibitors include, but are not limited to, VB-201 (a small molecule inhibitor of CD14).

Embodiments include methods of treating NPC or delaying the onset of NPC in a subject by administering an anti-TLR antibody. Examples of anti-TLR antibodies include, but are not limited to, OPN-305 (an anti-TLR2 antibody) and T2.5 (an anti-TLR2 antibody), NI-0101 (an anti-TLR4 antibody), 1A6 (an anti-TLR4/MD2 antibody). Examples of anti-CD14 antibodies include, but are not limited to, IC14.

Embodiments include methods of treating NPC or delaying the onset of NPC in a subject by administering an anti-TLR oligonucleotide. Examples of anti-TLR oligonucleotides include, but are not limited to, IRS-954 (an anti-TLR7/9 oligonucleotide), DV-1179 (an anti-TLR7/9 oligonucleotide), IMO-3100 (an anti-TLR7/9 oligonucleotide), IHN-ODN-24888 (an anti-TLR7/9 oligonucleotide), IMO-8400 (an anti-TLR7/8/9 oligonucleotide), IMO-9200 (an anti-TLR7/8/9 oligonucleotide), and IHN-ODN 2088 (an anti-TLR9 oligonucleotide).

Embodiments include methods of treating NPC or delaying the onset of NPC in a subject by administering an anti-TLR lipid A analog. In an embodiment, the anti-TLR lipid A analog is Eritoran (E5564), a lipid A analog inhibitor of TLR4/1VD2.

Embodiments include methods of treating NPC or delaying the onset of NPC in a subject by administering an anti-TLR miRNA. In an embodiment, the anti-TLR miRNA is an miRNA inhibitor of TLR4, such as miR-146a or miR-21.

Embodiments include methods of treating NPC or delaying the onset of NPC in a subject by administering an anti-TLR nano drug. Examples of anti-TLR nano drugs include, but are not limited to, non-anticoagulant heparin nanoparticle (NAHNP) (an anti-TLR4 nano drug), high-density lipoprotein-like nanoparticle (HDL-like NP) (an anti-TLR4 nano drug), bare gold nanoparticle (Bare GNP) (an anti-TLR4 nano drug), glycolipid-coated gold nanoparticle (an anti-TLR4/MD2 nano drug), and peptide-gold-nanoparticle hybrid P12 (an anti-TLR2/3/4/5 nano drug).

Embodiments include methods of treating NPC or delaying the onset of NPC in a subject by administering immunosuppressive drugs that target T-cell activation and/or T-cell interaction. Examples of such immunosuppressive drugs include, but are not limited to, calcineurin inhibitors (e.g., tacrolimus (FK506), cyclosporin A, and voclosporin), anti-TCR agents (e.g., TOL101, ChAglyCD3, and hOKT3 g1(Ala-Ala)), CTLA4-Ig (CD80/86 competitive inhibitor, e.g., abatacept and belatacept), anti-CD40 mAb (e.g., ASKP1240), anti-CD52 mAb (e.g., Alemtuzumab), and anti-LFA-1 mAb (e.g., efalizumab).

Embodiments include methods of treating NPC or delaying the onset of NPC in a subject by administering immunosuppressive drugs that target T-cell differentiation/proliferation and/or T-cell related cytokine production. Examples of such immunosuppressive drugs include, but are not limited to, methotrexate, mTOR inhibitors (e.g., sirolimus and everolimus), j anus kinase inhibitor (e.g., tofacitinib), antiproliferative agents (e.g., mycophenolate mofetil (CellCept®)), mycophenolate sodium, azathioprine, steroids (e.g., prednisone and corticosteroids), TNFα inhibitor (e.g., anti-TNFα mAb (Infliximab, adalimumab, golimumab, and certolizumab), TNFR inhibitor (e.g., TNFR-Ig (Etanercept)), IL-2R inhibitor (e.g., anti-IL-2R mAb (basiliximab)), anti-IL-17 mAb (e.g., secukinumab), and anti-IL-6 mAb (e.g., tocilizumab).

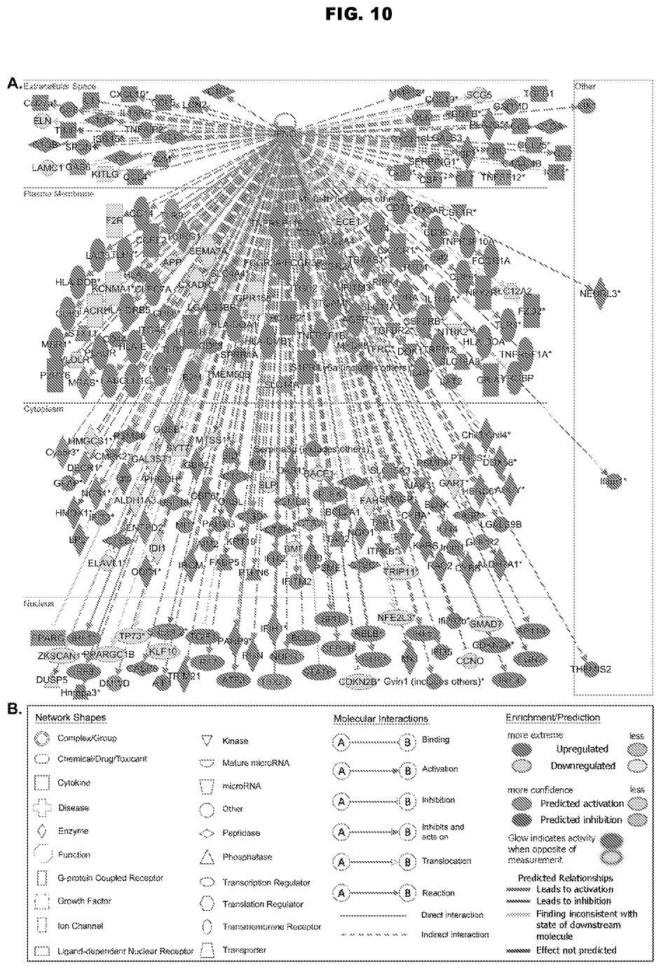

As discussed herein, increased neuroinflammation, marked by increased cerebellar astrocytosis as a result of Amyloid Precursor Protein (APP) loss of function, leads to an accelerated neurodegenerative phenotype. As demonstrated herein, genome-wide transcriptome analysis was performed using the cerebellar tissue samples from the following genotypes: Npc1^{+/+}/App^{+/+}, Npc1^{+/+}/App^{-/-}, Npc1^{-/-}/App^{+/+}, and Npc1^{-/-}/App^{-/-}, The results showed that the loss of APP function via App gene knockout resulted in exacerbation of the inflammatory pathways previously identified in NPC, such as the activation of microglia, antiviral response, activation of T-lymphocytes, and chemotaxis of T-lymphocytes (FIGS. 10 and 15-19). In FIG. 10A, comparative cerebellar transcriptome analysis (Npc1^{-/-}/App^{-/-} vs. Npc1^{+/+}/App^{+/+}) showed that interferon-gamma downstream signaling is severely exacerbated in Npc1^{-/-}/App^{-/-} mice, involving a total of 262 IFN-γ downstream genes. Previously, Npc1^{-/-}/App^{+/+} vs. Npc1^{+/+}/App^{+/+} comparison identified the differential expression of 60 IFN-γ downstream genes.

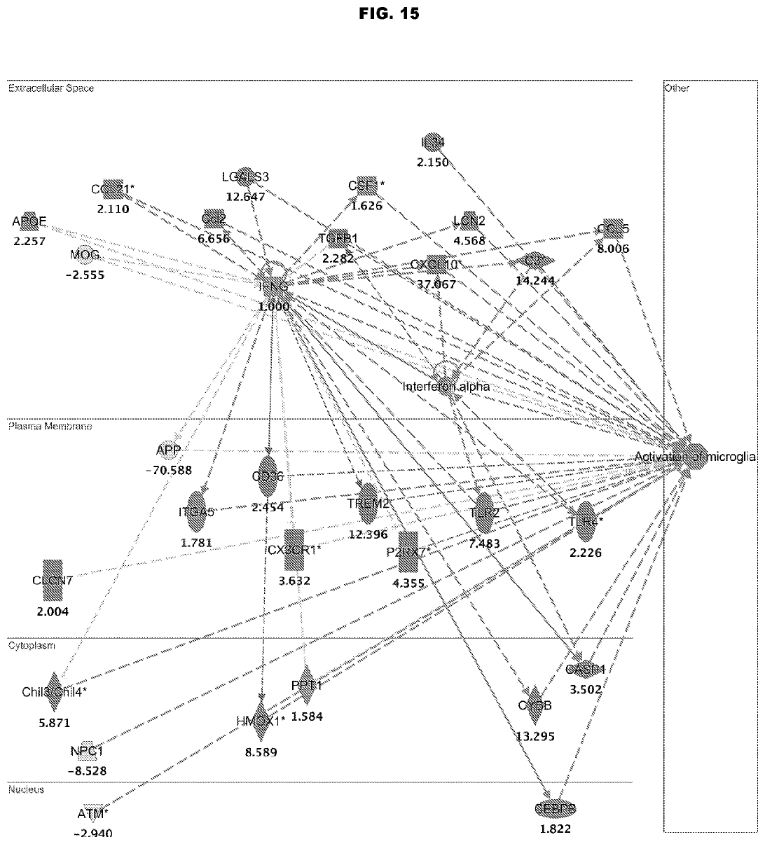

Loss of APP function results in the exacerbation of DEGs functionally related to the activation of microglia in Npc1−/−/App−/− mouse cerebella. In FIG. 15, a total of 29 genes related to microglial activation pathway were differentially expressed in the pre-symptomatic Npc1^{-/-}/App^{-/-} mouse cerebella compared to wild-type (Npc1^{+/+}/App^{+/+}). Of these, 25 were IFN-γ-responsive genes and 7 were IFN-α-responsive genes. All differentially expressed genes (DEGs) are localized to their sub-cellular location. All plotted DEGs meet the significance cutoff of fold-change (absolute FC>1.5) and p-value (p<0.05). *Duplicate identifiers used for the same gene. A detailed key for IPA molecular shape, color, and interaction is provided in FIG. 10B.

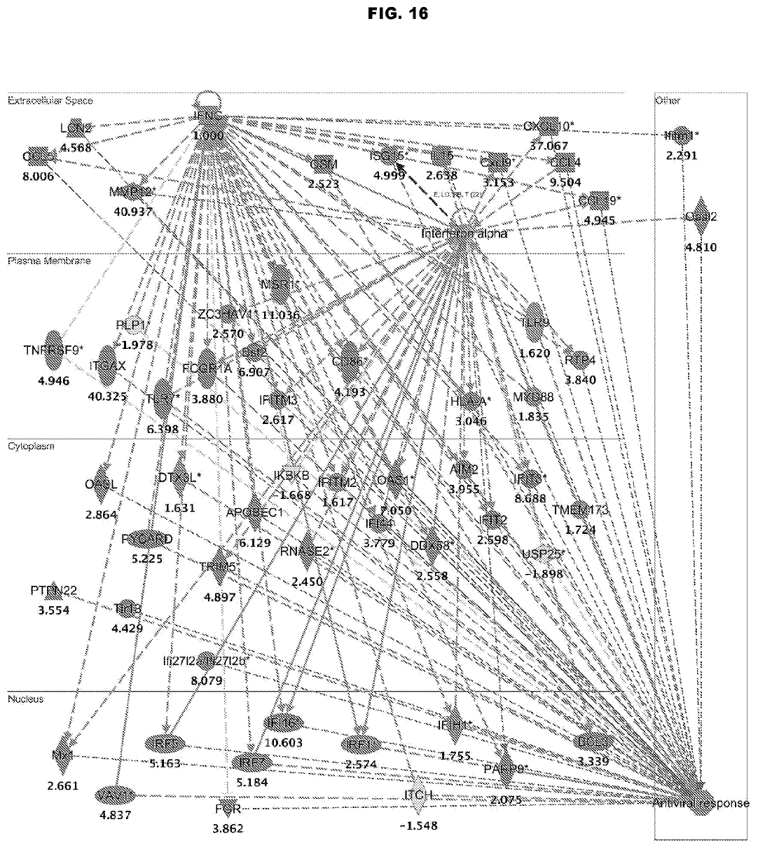

Loss of APP function results in the exacerbation of DEGs functionally related to antiviral response in Npc1−/−/App−/− mouse cerebella. In FIG. 16, a total of 56 genes related to antiviral response were differentially expressed in the pre-symptomatic Npc1^{-/-}/App^{-/-} mouse cerebella compared to wild-type (Npc1^{+/+}/App^{+/+}). Of these, 47 were IFN-γ-responsive genes and 39 were IFN-α-responsive genes. All differentially expressed genes (DEGs) are localized to their sub-cellular location. All plotted DEGs meet the significance cutoff of fold-change (absolute FC>1.5) and p-value (p<0.05). *Duplicate identifiers used for the same gene. A detailed key for IPA molecular shape, color, and interaction is provided in FIG. 10B.

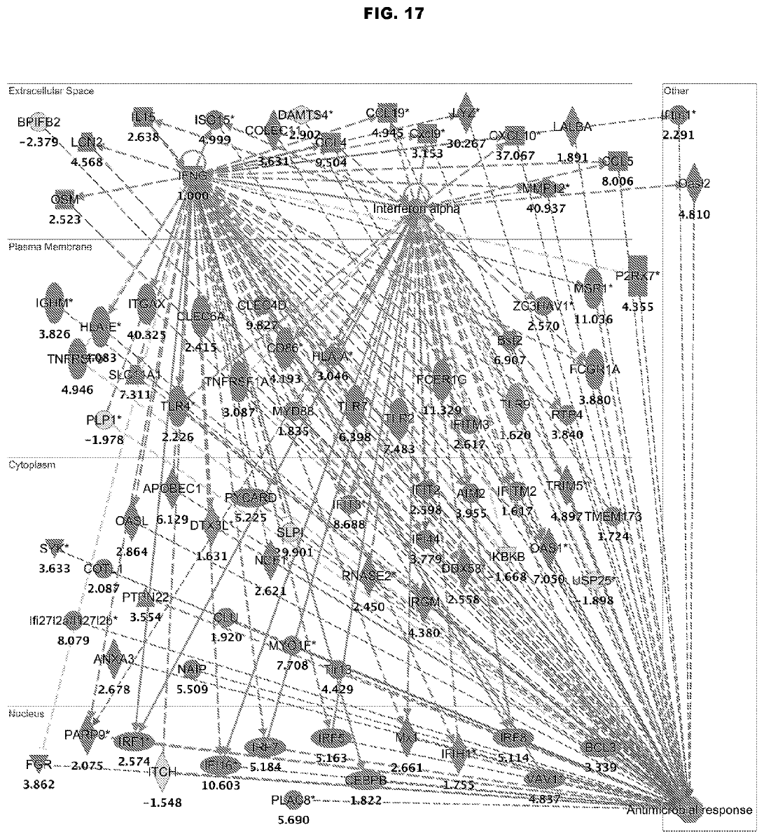

Loss of APP function results in the activation of the antimicrobial response pathway in Npc1−/−/App−/− mouse cerebella. In FIG. 17, a total of 87 genes related to activation of T-lymphocytes were differentially expressed in the pre-symptomatic Npc1^{-/-}/App^{-/-} mouse cerebella compared to wild-type (Npc1^{+/+}/App^{+/+}). Of these, 77 were IFN-γ-responsive genes and 34 were IFN-α-responsive genes. In Npc1−/−/App−/− mouse cerebella, 83 genes related to antimicrobial response were differentially expressed when compared with wild-type littermates (Npc1+/+/App+/+). IPA Upstream Analysis further identified that 62 of these genes are IFN-γ-responsive and 44 are identified to be IFN-α-responsive. All differentially expressed genes (DEGs) are localized to their sub-cellular location. All plotted DEGs meet the significance cutoff of fold-change (absolute FC>1.5) and p-value (p<0.05). *Duplicate identifiers used for the same gene. A detailed key for IPA molecular shape, color, and interaction is provided in FIG. 10B.

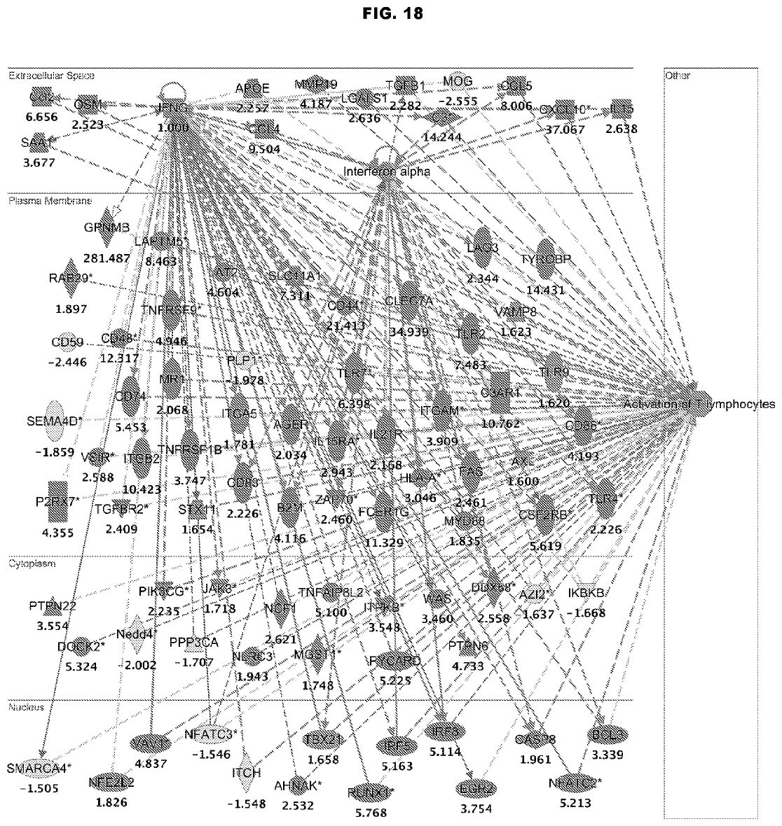

Loss of APP function results in the exacerbation of DEGs functionally related to the activation of T-lymphocytes in Npc1−/−/App−/− mouse cerebella. In FIG. 18, a total of 25 genes related to chemotaxis of T-lymphocytes were differentially expressed in the pre-symptomatic Npc1^{-/-}/App^{-/-} mouse cerebella compared to wild-type (Npc1^{+/+}/App^{+/+}). Of these, 18 were IFN-γ-responsive genes and 8 were IFN-α-responsive genes. All differentially expressed genes (DEGs) are localized to their sub-cellular location. All plotted DEGs meet the significance cutoff of fold-change (absolute FC>1.5) and p-value (p<0.05). *Duplicate identifiers used for the same gene. A detailed key for IPA molecular shape, color, and interaction is provided in FIG. 10B.

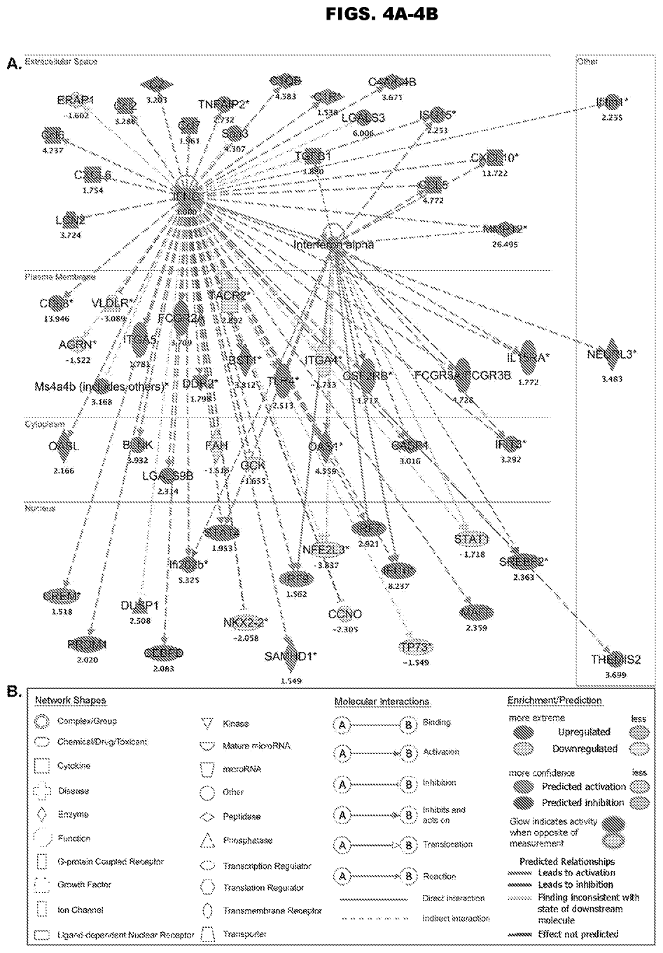

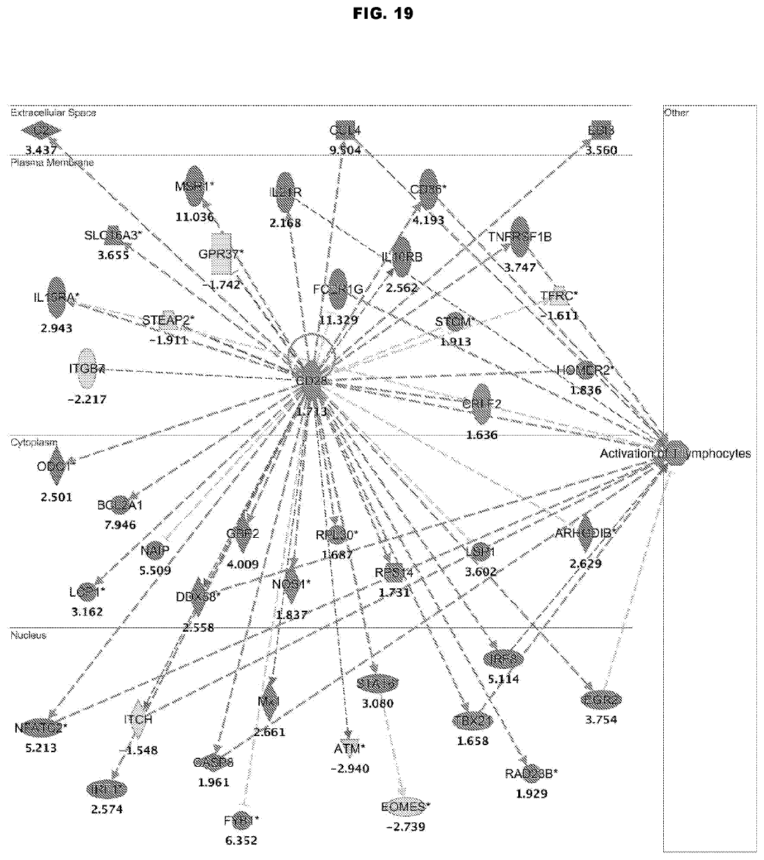

There is activation of T-lymphocyte co-stimulatory receptor CD28 in Npc1−/−/App−/− mouse cerebella (FIG. 19). All differentially expressed genes (DEGs) are localized to their sub-cellular location. All plotted DEGs meet the significance cutoff of fold-change (absolute FC>1.5) and p-value (p<0.05). *Duplicate identifiers used for the same gene. A detailed key for IPA molecular shape, color, and interaction is provided in FIG. 4B.

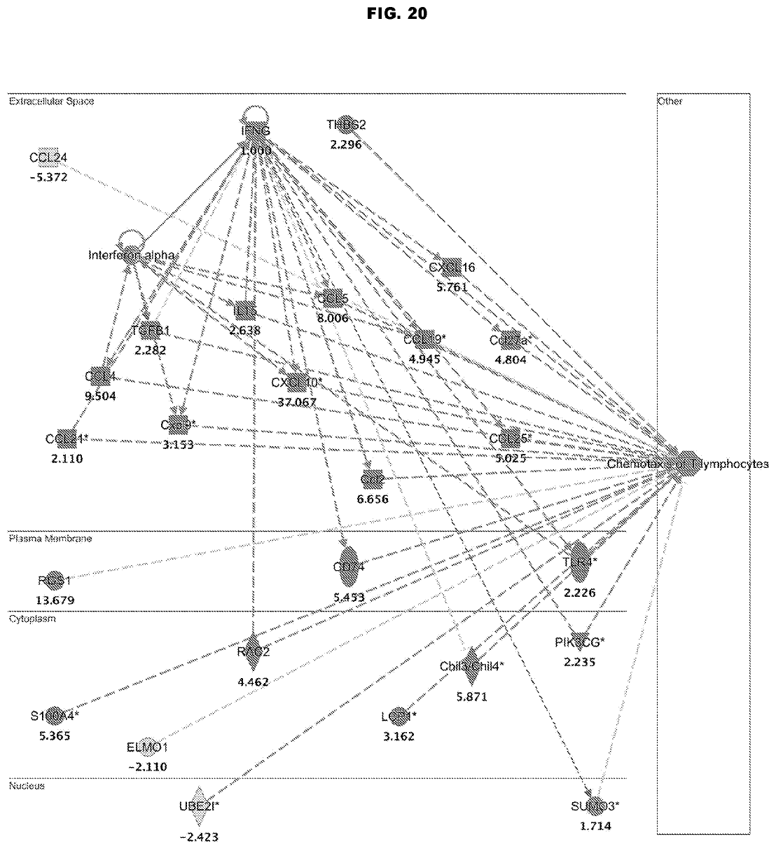

Loss of APP function results in the exacerbation of DEGs functionally related to the chemotaxis of T-lymphocytes in Npc1−/−/App−/− mouse cerebella (FIG. 20). All differentially expressed genes (DEGs) are localized to their sub-cellular location. All plotted DEGs meet the significance cutoff of fold-change (absolute FC>1.5) and p-value (p<0.05). *Duplicate identifiers used for same gene. A detailed IPA key for molecular shape, color and interaction is provided in FIG. 4B.

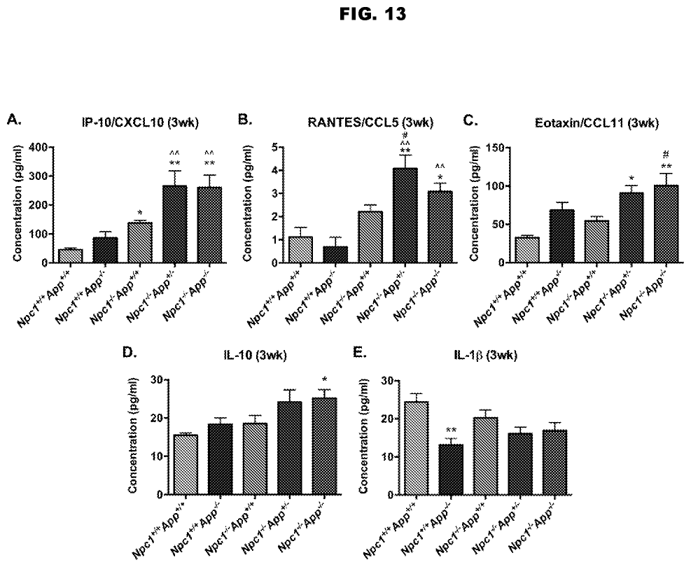

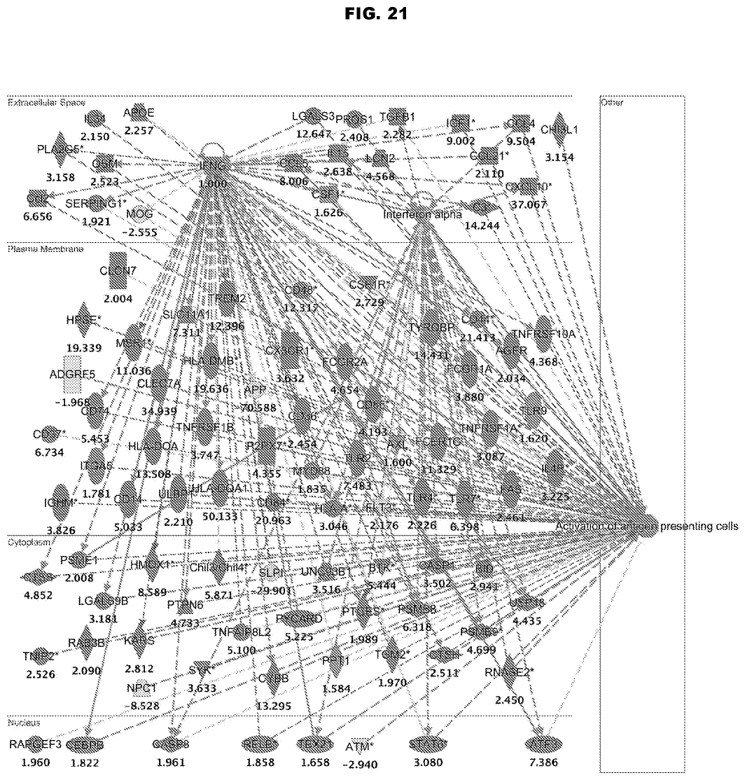

In addition, loss of APP function resulted in the activation of the antigen presentation pathway (FIG. 21). In FIG. 21, a total of 30 genes related to antigen presentation were differentially expressed in the pre-symptomatic Npc1^{-/-}/App^{-/-} mouse cerebella compared to wild-type (Npc1^{+/+}/App^{+/+}). All differentially expressed genes (DEGs) are localized to their sub-cellular location. All plotted DEGs meet the significance cutoff of fold-change (absolute FC>1.5) and p-value (p<0.05). *Duplicate identifiers used for the same gene. A detailed key for IPA molecular shape, color, and interaction is provided in FIG. 4B. Of these, 28 were IFN-γ-responsive genes and 14 were IFN-α-responsive genes. Protein levels of key inflammatory cytokines and chemokines were assessed in pre-symptomatic and terminal stage mouse cerebella from the following genotypes: Npc1^{+/+}/App^{+/+}, Npc1^{+/+}/App^{-/-}, Npc1^{-/-}/App^{+/+}, Npc1^{-/-}/App^{+/-}, and Npc1^{-/-}/App^{-/-}. As demonstrated herein, at pre-symptomatic stages, loss of APP in NPC mice more than doubles the increase in the expression of IP10/CXCL10 (FIGS. 13A-13E). Progressive loss of functional App allele (Npc1^{-/-}/App^{+/-} and Npc1^{-/-}/App^{-/-}) in NPC mouse model (Npc1^{-/-}/App^{+/+}) resulted in significant increase of pro-inflammatory cytokines at 3 weeks of age. Cytokines were measured by Multiplexed magnetic bead-based immunoassay kit (Catalog #MCYTMAG-70K-PX32, Millipore Sigma, Burlington Mass.). As shown in FIG. 13A, IFN-γ-responsive cytokine IP-10/CXCL10 is the only protein significantly increased in Npc1^{-/-}/App^{+/+} in the pre-symptomatic mouse cerebella. This increased expression is significantly exacerbated with the loss of APP function (compare Npc1^{-/-}/App^{-/-} with Npc1^{-/-}/App^{+/-} and Npc1^{-/-}/App^{-/-}). MIG/CXCL9, RANTES/CCL5, EOTAXIN/CCL11, and IL-10 (FIGS. 13B-13D) were also significantly increased in Npc1^{-/-}/App^{-/-} and/or Npc1^{-/-}/App^{-/-} mouse cerebella compared to wild-type (Npc1^{+/+}/App^{+/+}) and/or Npc1^{-/-}/App^{+/+}. FIG. 13E is a graphical representation of the expression of IL-1β expression in Npc1^{-/-}/App^{+/-} and/or Npc1^{-/-}/App^{-/-} mouse cerebella compared with wild-type (Npc1^{+/+}/App^{+/+}) and/or Npc1^{-/-} /App^{+/+}. Values are means±SEM. *p<0.05, **p<0.01, ***p<0.001, ****p<0.0001. *=compared to Npc1^{+/+}/App^{+/+}; \hat{ }=compared to Npc1^{+/+}/App^{-/-}; #=compared to Npc1^{-/-}/App^{+/+}. Moreover, in the most widely used Npc1^{-/-} mice (BALB/cNctr-Npc1^{miN}/J), microglial activation and reactive astrocytosis have been reported as early as 2 weeks of age, significantly prior to the typical onset of neurological deficits around 7-8 weeks of age observed in this strain.

In addition, prior to disease onset, the following inflammatory agents are also increased: MIG/CXCL9, RANTES/CCL5, EOTAXIN/CCL11, and IL-10. The dysregulation of these inflammatory agents is directly linked to the loss of APP function (see, e.g., FIGS. 13A-13E). Further, neuroinflammation is not only present in early NPC disease but also contributes directly to NPC neurodegeneration. Thus, loss of APP function activates, exacerbates, and accelerates disease onset and neurodegenerative phenotype and decreases life expectancy in NPC mice. Therapies that prevent or reduce APP loss of function can be used to treat NPC in a subject or delay the onset of NPC in a subject.

Furthermore, modulation of the activity of the APP gene to optimize their expression can be used as a therapeutic strategy to treat NPC in a subject or delay the onset of NPC in a subject. Studies have shown that the secreted domain of the APP protein (sAPPalpha) is responsible for most of its neuroprotective function. As a therapeutic strategy for NPC, in some embodiments, the compounds RS67333 and donecopride can be used to treat NPC in a subject or delay the onset of NPC in a subject. Both are partial serotonin subtype 4 receptor agonists and additionally promote the generation of sAPPalpha with comparable profiles.

In some embodiments, one or more molecules or agents that can protect against IP10/CXCL10 mediated apoptosis can be used to treat NPC in a subject or delay the onset of NPC in a subject. For example, neuregulin-1 (NRG-1) protects against IP10/CXCL10 mediated apoptosis and can be used to treat NPC in a subject or delay the onset of NPC in a subject.

Moreover, one or more fatty acid binding protein (FABP) inhibitors can be used to treat NPC in a subject or delay the onset of NPC in a subject. FABP4 mediates lipid-dysregulation induced microglial activation and neuroinflammation. Members of the FABP family, including FABP3, FABP5, and FABP7, have altered expression in the NPC1 mutant cerebellum relative to control. Examples of FABP inhibitors that can be used to treat NPC in a subject or delay the onset of NPC in a subject include, but are not limited to, e.g., BMS309403 (an FABP4 inhibitor) and HTS01037.

Further, TLR4 was identified in the IPA disease and function analysis as an IFN-γ-responsive gene that is directly related to the activation of microglia and is differentially expressed in the early NPC cerebella (see, e.g., FIG. 4). The activation of TLR4 leads to sphingosine kinase 1 activation and subsequent increase in sphingosine 1 phosphate (SIP). S1P is a bioactive lipid that binds S1P receptor (S1PR) and promote lymphocyte egress from lymphoid tissue to the site of inflammation. Moreover, S1P binding to S1PR also induce IP10/CXCL10 release from astrocytes. Thus, agents that downregulate S1PR and inhibit lymphocyte egress are beneficial to treat or delay NPC as T-lymphocyte activation and chemotaxis are strongly implicated. An example of such an agent is FTY720 (Fingolimod®). FTY720 may also directly inhibit the aberrant increase in IP10/CXCL10 in the NPC brains. In some embodiments, FTY720 can be used to treat NPC in a subject or delay the onset of NPC in a subject.

In some embodiments, one or more inhibitors of MCP1/CCL2 can be used to treat NPC in a subject or delay the onset of NPC in a subject. Examples of inhibitors of MCP1/CCL2 include, but are not limited to, bindarit (2-methyl-2-((1-(phenylmethyl)-1H-indazol-3yl) methoxy) propanoic acid) (an inhibitor of MCP1/CCL2 synthesis), spiegelmer (mNOX-E36), MCP-1(9-76) (an MCP1 antagonist), 7ND (via plasmid, as an MCP1 inhibitor (MCP1 mutant), anti-MCP1 antibodies (such as anti-human CCL2 mAb (Carlumab; clone ID CNTO 888) and C775 (as described in U.S. Pat. No. 7,371,825)), miR-124, insulin, paraoxonase-1, heme oxygenase-1, NS-398 inhibitor of cyclooxygenase-2, trichostatin A (inhibitor/histone deacetylases), quercetin (3,3′,4′,5,7-pentahydroxyflavone), tat-C3 exoenzyme, dominant-negative RhoA, FR 167653 (p38 MAPK inhibitor), SB 203580 (p38 MAPK inhibitor), PD 98059 (ERK), AG 490 (JAK-2), pyrrolidine dithiocarbamate (potent antioxidant and an inhibitor of NF-κB), doxycycline, minocycline, doxazosin, vMIP-II, montelukast and zafirlukas (leukotriene receptor antagonists (LTRAs), calcium channel blockers (amlodipine and manidipine), irbesartan, rosiglitazone, troglitazone, pioglitazone, pravastatin, cerivastatin, simvastatin, atorvastatin, aspirin, fenofibrate, and clofibrate.

In some embodiments, one or more inhibitors of CCR2 can be used to treat NPC in a subject or delay the onset of NPC in a subject. Examples of inhibitors of CCR2 include, but are not limited to, propagermanium (a CCR2 (MCP1 receptor) antagonist), 15a, AZ889, RAP-103 (a potent antagonist of both CCR2 (IC50=4.2 pM) and CCR5 (IC50=0.18 pM) mediated monocyte chemotaxis), PF-04136309 (Pfizer), and MCPR-04, MCPR-05, and MCPR-06.

In some embodiments, one or more inhibitors of MIP-1α/CCL3 can be used to treat NPC in a subject or delay the onset of NPC in a subject. Examples of inhibitors of MIP-1α/CCL3 include, but are not limited to, adenosine receptor antagonists (such as N6-(3-iodobenzyl)-adenosine-5′-N-methyluronamide (IB-MECA) and 2-p-(2-carboxyethyl) phenethylamino5′-N-ethyl-carboxamidoadenosine (CGS)), evasin-1 (a chemokine binding protein), trichostatin A (an inhibitor/histone deacetylase), and miR-223.

In some embodiments, one or more inhibitors of MIP-1β/CCL4 can be used to treat NPC in a subject or delay the onset of NPC in a subject. Examples of inhibitors of MIP-1β/CCL4 include, but are not limited to, monoclonal antibody against CCL4 (Clone ID 24006, available from multiple sources), microRNA-195 (an anti-MIP-1β/CCL4), and miR-125b.

In some embodiments, one or more inhibitors of IL-1α can be used to treat NPC in a subject or delay the onset of NPC in a subject. Examples of inhibitors of IL-1α include, but are not limited to, anakinra (a receptor antagonist for IL-1RI, Swedish Orphan BioVitrum), rilonacept (a soluble IL-1 receptor that binds IL-1β>IL-1α>IL-1Ra, Regeneron), canakinumab, gevokizumab, LY2189102, anti-IL-1α mAb, anti-IL-1 receptor mAb, oral caspase 1 inhibitors, MABp1 (neutralizing anti-IL-1α IgG1 mAb,)(Biotech), MEDI-8968 (a blocking antibody to IL-1RI, MedImmune), and VX-765 (an oral caspase 1 inhibitor, Vertex Pharmaceuticals).

In some embodiments, one or more inhibitors of KC/CXCL1 can be used to treat NPC in a subject or delay the onset of NPC in a subject. Examples of inhibitors of KC/CXCL1 include, but are not limited to, monoclonal antibodies, such as Clone ID 48415 (as described in Parkunan et al. 2016) and Clone ID HL2401 (as described in Miyake et al. 2019).

In some embodiments, one or more inhibitors of IFIT3 can be used to treat NPC in a subject or delay the onset of NPC in a subject. Examples of inhibitors of IFIT3 include, but are not limited to, monoclonal antibody clone ID OTI1G1.

In some embodiments of the disclosure, any of the inhibitors described above, e.g., an IFN inhibitor (e.g., a Type I IFN inhibitor or a Type II IFN inhibitor), an IP10/CXCL10 inhibitor, a CXCR3 inhibitor, an FABP inhibitor, or an inhibitor of any one of the inflammatory agents MIG/CXCL9, RANTES/CCL5, EOTAXIN/CCL11, and IL-10 can be an inhibitory RNA (e.g., an antisense RNA, small interfering RNA (siRNA), microRNA (miRNA), or short hairpin RNA (shRNA)). In some embodiments, the inhibitory RNA targets a sequence that is identical or substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a target sequence in a target polynucleotide (e.g., a portion comprising at least 20, at least 30, at least 40, at least 50, at least 60, at least 70, at least 80, at least 90, or at least 100 contiguous nucleotides, e.g., from 20-500, 20-250, 20-100, 50-500, or 50-250 contiguous nucleotides of the target polynucleotide sequence). For example, an inhibitory RNA can target a sequence that is identical or substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a target sequence in a target polynucleotide encoding an IFN (e.g., the sequence of GenBank ID No. NM_000605.3 encoding IFN-α, the sequence of GenBank ID No. NM_002176.4 encoding IFN-β, or the sequence of GenBank ID No. NM_000619.3), a target polynucleotide encoding an IP10/CXC10 (e.g., the sequence of GenBank ID No. NM_001565.4), a target polynucleotide encoding a CXCR3 (e.g., the sequence of GenBank ID No. NM_001504.2), a target polynucleotide encoding an FABP (e.g., the sequence of GenBank ID No. NM_001442.2 encoding FABP4), a target polynucleotide encoding any one of the inflammatory agents MIG/CXCL9 (e.g., the sequence of GenBank ID No. NM_002416.2), RANTES/CCL5 (e.g., the sequence of GenBank ID No. NM_002985.2), EOTAXIN/CCL11 (e.g., the sequence of GenBank ID No. NM_002986.2), and IL-10 (e.g., the sequence of GenBank ID No. NM_000572.3), or a target polynucleotide encoding any one of the inflammatory agents MCP1/CCL2 (e.g., the sequence of GenBank ID No. NM_002982.4), MIP-1α/CCL3 (e.g., the sequence of GenBank ID No. NM_002983.3), MIP-1β/CCL4 (e.g., the sequence of GenBank ID No. NM_002984.4), IL-1α (e.g., the sequence of GenBank ID No. NM_000575.4), and KC/CXCL1 (e.g., the sequence of GenBank ID No. NM_001511.3). In particular embodiments, an inhibitory RNA can target a sequence that is identical or substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a target sequence in a target polynucleotide encoding an IP10/CXC10 (e.g., the sequence of GenBank ID No. NM_001565.4).

In some embodiments, the disclosure includes treating NPC in a subject or delaying the onset of NPC in a subject using an shRNA or siRNA. An shRNA is an artificial RNA molecule with a hairpin turn that can be used to silence target gene expression via the siRNA it produces in cells. Expression of shRNA in cells is typically accomplished by delivery of plasmids or through viral or bacterial vectors. Suitable bacterial vectors include but not limited to adeno-associated viruses (AAVs), adenoviruses, and lentiviruses. After the vector has integrated into the host genome, the shRNA is then transcribed in the nucleus by polymerase II or polymerase III (depending on the promoter used). The resulting pre-shRNA is exported from the nucleus, then processed by Dicer and loaded into the RNA-induced silencing complex (RISC). The sense strand is degraded by RISC and the antisense strand directs RISC to an mRNA that has a complementary sequence. A protein called Ago2 in the RISC then cleaves the mRNA, or in some cases, represses translation of the mRNA, leading to its destruction and an eventual reduction in the protein encoded by the mRNA. Thus, the shRNA leads to targeted gene silencing. In some embodiments, a method of treating NPC in a subject or delaying the onset of NPC in a subject comprises administering to the subject a therapeutically effective amount of a vector comprising a polynucleotide that encodes an shRNA capable of hybridizing to a portion of a target polynucleotide encoding an IFN, a target polynucleotide encoding an IP10/CXC10 (e.g., the sequence of GenBank ID No. NM_001565.4), a target polynucleotide encoding a CXCR3 (e.g., the sequence of GenBank ID No. NM_001504.2), a target polynucleotide encoding an FABP (e.g., the sequence of GenBank ID No. NM_001442.2 encoding FABP4), a target polynucleotide encoding any one of the inflammatory agents MIG/CXCL9 (e.g., the sequence of GenBank ID No. NM_002416.2), RANTES/CCL5 (e.g., the sequence of GenBank ID No. NM_002985.2), EOTAXIN/CCL11 (e.g., the sequence of GenBank ID No. NM_002986.2), and IL-10 (e.g., the sequence of GenBank ID No. NM_000572.3), or a target polynucleotide encoding any one of the inflammatory agents MCP1/CCL2 (e.g., the sequence of GenBank ID No. NM_002982.4), MIP-1α/CCL3 (e.g., the sequence of GenBank ID No. NM_002983.3), MIP-1β/CCL4 (e.g., the sequence of GenBank ID No. NM_002984.4), IL-1α (e.g., the sequence of GenBank ID No. NM_000575.4), and KC/CXCL1 (e.g., the sequence of GenBank ID No. NM_001511.3). In particular embodiments, a method of treating NPC in a subject or delaying the onset of NPC in a subject comprises administering to the subject a therapeutically effective amount of a vector comprising a polynucleotide that encodes an shRNA capable of hybridizing to a portion of a target polynucleotide encoding an IP10/CXC10 (e.g., the sequence of GenBank ID No. NM_001565.4).

In some embodiments, the disclosure comprises treating NPC in a subject or delaying the onset of NPC in a subject using a microRNA (miRNA or miR). A microRNA is a small non-coding RNA molecule that functions in RNA silencing and post-transcriptional regulation of gene expression. miRNAs base pair with complementary sequences within the mRNA transcript. As a result, the mRNA transcript may be silenced by one or more of the mechanisms such as cleavage of the mRNA strand, destabilization of the mRNA through shortening of its poly(A) tail, and decrease translation efficiency of the mRNA transcript into proteins by ribosomes. In some embodiments, a method of treating NPC in a subject or delaying the onset of NPC in a subject comprises administering to the subject a therapeutically effective amount of a vector comprising a polynucleotide that encodes a miRNA capable of hybridizing to a portion of a target polynucleotide encoding an IFN, a target polynucleotide encoding an IP10/CXC10 (e.g., the sequence of GenBank ID No. NM_001565.4), a target polynucleotide encoding a CXCR3 (e.g., the sequence of GenBank ID No. NM_001504.2), a target polynucleotide encoding an FABP (e.g., the sequence of GenBank ID No. NM_001442.2 encoding FABP4), a target polynucleotide encoding any one of the inflammatory agents MIG/CXCL9 (e.g., the sequence of GenBank ID No. NM_002416.2), RANTES/CCL5 (e.g., the sequence of GenBank ID No. NM_002985.2), EOTAXIN/CCL11 (e.g., the sequence of GenBank ID No. NM_002986.2), and IL-10 (e.g., the sequence of GenBank ID No. NM_000572.3), or a target polynucleotide encoding any one of the inflammatory agents MCP1/CCL2 (e.g., the sequence of GenBank ID No. NM_002982.4), MIP-1α/CCL3 (e.g., the sequence of GenBank ID No. NM_002983.3), MIP-1β/CCL4 (e.g., the sequence of GenBank ID No. NM_002984.4), IL-1α (e.g., the sequence of GenBank ID No. NM_000575.4), and KC/CXCL1 (e.g., the sequence of GenBank ID No. NM_001511.3). In particular embodiments, a method of treating NPC in a subject or delaying the onset of NPC in a subject comprises administering to the subject a therapeutically effective amount of a vector comprising a polynucleotide that encodes a miRNA capable of hybridizing to a portion of a target polynucleotide encoding an IP10/CXC10 (e.g., the sequence of GenBank ID No. NM_001565.4).

In some embodiments, the disclosure comprises treating NPC in a subject or delaying the onset of NPC in a subject using an antisense oligonucleotide, e.g., an RNase H-dependent antisense oligonucleotide (ASO). ASOs are single-stranded, chemically modified oligonucleotides that bind to complementary sequences in target mRNAs and reduce gene expression both by RNase H-mediated cleavage of the target RNA and by inhibition of translation by steric blockade of ribosomes. In some embodiments, a method of treating NPC in a subject or delaying the onset of NPC in a subject comprises administering to the subject a therapeutically effective amount of a vector comprising a polynucleotide that encodes an ASO capable of hybridizing to a portion of a target polynucleotide encoding an IFN, a target polynucleotide encoding an IP10/CXC10 (e.g., the sequence of GenBank ID No. NM_001565.4), a target polynucleotide encoding a CXCR3 (e.g., the sequence of GenBank ID No. NM_001504.2), a target polynucleotide encoding an FABP (e.g., the sequence of GenBank ID No. NM_001442.2 encoding FABP4), a target polynucleotide encoding any one of the inflammatory agents MIG/CXCL9 (e.g., the sequence of GenBank ID No. NM_002416.2), RANTES/CCL5 (e.g., the sequence of GenBank ID No. NM_002985.2), EOTAXIN/CCL11 (e.g., the sequence of GenBank ID No. NM_002986.2), and IL-10 (e.g., the sequence of GenBank ID No. NM_000572.3), or a target polynucleotide encoding any one of the inflammatory agents MCP1/CCL2 (e.g., the sequence of GenBank ID No. NM_002982.4), MIP-1α/CCL3 (e.g., the sequence of GenBank ID No. NM_002983.3), MIP-1β/CCL4 (e.g., the sequence of GenBank ID No. NM_002984.4), IL-1α (e.g., the sequence of GenBank ID No. NM_000575.4), and KC/CXCL1 (e.g., the sequence of GenBank ID No. NM_001511.3). In particular embodiments, a method of treating NPC in a subject or delaying the onset of NPC in a subject comprises administering to the subject a therapeutically effective amount of a vector comprising a polynucleotide that encodes an ASO capable of hybridizing to a portion of a target polynucleotide encoding an IP10/CXC10 (e.g., the sequence of GenBank ID No. NM_001565.4).

### EXAMPLES

Various examples are provided to illustrate selected aspects of the various embodiments.

### Example 1: Methods and Experimental Procedures

Mice and Tissue Processing. A colony of BALB/cNctr-Npc1^{miN}/J mice was established and maintained in the Loma Linda University Animal Care Facility (LLUACF) according to the Institutional Animal Care and Use Committee (IACUC) approved protocol and NIH guidelines. Breeding pairs of BALB/cNctr-Npc1^{miN}/J mice heterozygous for the recessive NIH allele of the Niemann-Pick Type C1 gene were obtained from the Jackson Laboratory and bred in-house at LLUACF to generate wild-type (Npc1^{+/+}) and homozygous Npc1 knockout (Npc1^{-/-}) genotypes. The mice were given free access to water and food. For the Npc1^{-/-} mice that began to display motor dysfunction, chow and hydrogel were provided directly on the bedding to facilitate access. Mice were identified by metal ear tags and genotypes were determined by PCR analysis of genomic DNA. Tissue samples were collected according to the approved LLU IACUC protocol. Briefly, under deep isoflurane anesthesia, transcardial perfusion was followed by a quick decapitation with a scalpel. Brains were extracted, cut sagittally in ice-cold PBS, and snap frozen in liquid nitrogen. Samples were stored in −80° C. until the time of analysis.

Mice lacking both APP and NPC1 proteins were generated. Briefly, breeding pairs of mice heterozygous for the recessive NIH allele of the Niemann-Pick Type C1 gene (BALB/cNctr-Npc1^{miN}/J) and homozygous knockout mice for the Amyloid Precursor Protein gene (B6.129S7-App^{tm1Dbo}/J) were obtained from the Jackson Laboratory and crossed to generate breeders that are double heterozygous for NPC1 and APP (Npc1^{+/-}/App^{het}) in the mixed BALB/c/B6.129S7 background. The double heterozygous breeding system was maintained in-house in the Loma Linda University Animal Care Facility according to the Institutional Animal Care and Use Committee (IACUC) approved protocol (LLU #8180006) and NIH guidelines to generate wild-type (Npc1^{+/+}/App^{wt}), single knockout (App^{ko} and Npc1^{-/-}), and double mutant (Npc1^{-/-}/App^{het} and Npc1^{-/-}/App^{ko}) mice for the transcriptome and protein analyses. All mice were given free access to water and food. For the mice lacking the NPC1 protein (Npc1^{-/-}, Npc1^{-/-}/App^{het}, and Npc1^{-/-}/App^{ko}) that began to display motor dysfunction, chow and HydroGel® (ClearH2O, Portland, Me.) were provided directly on the bedding to facilitate access. Mice were identified by metal ear tags and genotypes were determined by PCR analysis of genomic DNA. Tissue samples at were collected according to the approved LLU IACUC protocol #8180006. Under deep isoflurane anesthesia, transcardial perfusion was followed by a quick decapitation with a scalpel. Brains were extracted, cut sagittally in ice-cold PBS, snap frozen in liquid nitrogen, and stored in −80° C. until the time of analysis.

Cytokine Detection. The levels of 32 inflammatory cytokines in the cerebella of wild-type (Npc1^{+/+}) and NPC1 knockout (Npc1^{-/-}) mice were analyzed simultaneously using Milliplex 32-plex Mouse Cytokine/Chemokine Magnetic Bead Panel (Catalog #MCYTMAG-70K-PX32, Millipore Sigma, Burlington Mass.) according to the manufacturer's instructions. Briefly, the cerebella samples were thawed on ice, weighed, and homogenized in protein extraction buffer (Sterile PBS, 0.05% Triton X, Halt™ Protease Inhibitor Cocktail (Thermo Fisher Scientific, Waltham Mass.)) using acid-washed 1.4 mm zirconium beads and benchtop BeadBug™ tissue homogenizer (Benchmark Scientific, Sayreville, N.J.). Homogenates were sonicated for 1 minute in the sonication bath (Branson M1800, Branson Ultrasonics, Danbury, Conn.) and centrifuged at 10,000 g for 20 mins at 4° C., as previously described. For the assay panel, 25 μL of standard, quality control, and brain tissue protein samples were mixed with 25 μL of pre-mixed bead solution in a 96-well plate, sealed, and incubated at 4° C. overnight on a plate shaker. Subsequently, the plates were washed twice and 25 μL of detection antibodies were added to each well, sealed, light-protected, and incubated at room temperatures for 1 hour on a plate shaker. Lastly, 25 μL of Streptavidin-Phycoerythrin were added to each well, sealed, light-protected, and incubated at room temperature for 30 minutes on a plate shaker. Following the incubation, plates were washed twice according to manufacturer's protocol and 150 μL of MAGPIX® Drive Fluid was added to all wells and read on MAGPIX® (Luminex Corp., Austin Tex.). The data were analyzed using MasterPlex 2010 software (Hitachi Solutions America, San Bruno, Calif.). All data for cytokine analysis are represented as the mean±standard error. The statistical significance between the wild-type (Npc1^{+/+}) and NPC1 knockout (Npc1^{-/-}) samples were analyzed by two-tailed student's t-test with p-values<0.05 considered statistically significant. The 32 analyzed molecules were eotaxin (CCL11), granulocyte colony-stimulating factor (G-CSF), granulocyte-macrophage colony-stimulating factor (GM-CSF), interferon-gamma (IFN-γ), interleukin-1α (IL-1α), interleukin-10 (IL-1β), interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12/p40), interleukin-12 (IL-12/p70), interleukin-13 (IL-13), interleukin-15 (IL-15), interleukin-17 (IL-17), interferon gamma induced protein 10 (IP10/CXCL10), keratinocyte chemoattractant (KC/CXCL1), leukemia inhibitory factor (LIF), lipopolysaccharide-inducible CXC chemokine (LIX/CXCL5), monocyte chemoattractant protein-1 (MCP-1/CCL2), macrophage colony-stimulating factor (M-CSF), monokine induced by gamma interferon (MIG/CXCL9), macrophage inflammatory protein-1α (MIP-1α/CCL3), macrophage inflammatory protein-1β (MIP-1β/CCL4), macrophage inflammatory protein-2 (MIP-2/CXCL2), regulated on activation normal T cell expressed and secreted (RANTES/CCL5), tumor necrosis factor alpha (TNF-α), and vascular endothelial growth factor (VEGF).

The levels of 32 inflammatory cytokines and chemokines in the cerebella from Npc1^{+/+}/App^{+/+}, Npc1^{+/+}/App^{-/-}, Npc1^{-/-}/App^{+/+}, Npc1^{-/-}/App^{+/-}, and Npc1^{-/-}/App^{-/-} mice were simultaneously analyzed using Milliplex 32-plex Mouse Cytokine/Chemokine Magnetic Bead Panel (Catalog #MCYTMAG-70K-PX32, Millipore Sigma, Burlington Mass.) according to the manufacturer's instructions. Cerebellar tissue was homogenized in protein extraction buffer (PBS, 0.05% Triton X, Halt Protease Inhibitor Cocktail (Thermo Fisher Scientific, Waltham Mass.)) using acid-washed 1.4 mm zirconium beads and benchtop BeadBug™ tissue homogenizer (Benchmark Scientific, Sayreville, N.J.). Homogenates were sonicated for 1 minute in the sonication bath (Branson M1800, Branson Ultrasonics, Danbury, Conn.) and centrifuged at 10,000 g for 20 mins at 4° C. Multiplexed magnetic bead-based immunoassay kit was used according to the manufacturer's instructions. All data for cytokine/chemokine analyses are represented as the mean±standard error. One-way ANOVA and Tukey's post-hoc test were used to determine statistical significance between genotypes with p<0.05 considered significant. The 32 analyzed molecules were eotaxin (CCL11), granulocyte colony-stimulating factor (G-CSF), granulocyte-macrophage colony-stimulating factor (GM-CSF), interferon-gamma (IFN-γ), interleukin-1α (IL-1α), interleukin-1β(IL-1β), interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12/p40), interleukin-12 (IL-12/p′70), interleukin-13 (IL-13), interleukin-15 (IL-15), interleukin-17 (IL-17), interferon-gamma-induced protein 10 (IP-10/CXCL10), keratinocyte chemoattractant (KC/CXCL1), leukemia inhibitory factor (LIF), lipopolysaccharide-inducible CXC chemokine (LIX/CXCL5), monocyte chemoattractant protein-1 (MCP-1/CCL2), macrophage colony-stimulating factor (M-CSF), monokine induced by gamma interferon (MIG/CXCL9), macrophage inflammatory protein-1α (MIP-1α/CCL3), macrophage inflammatory protein-1β (MIP-1β/CCL4), macrophage inflammatory protein-2 (MIP-2/CXCL2), regulated on activation normal T cell expressed and secreted (RANTES/CCL5), tumor necrosis factor alpha (TNF-α), and vascular endothelial growth factor (VEGF). A total 45 cerebellar samples (3-week or terminal stage) from wild-type (Npc1^{+/+}/App^{+/+}), APP knockout (Npc1^{+/+}/App^{-/-}), NPC1 knockout (Npc1^{-/-}/App^{+/+}), NPC1 knockout/APP heterozygote (Npc1^{-/-}/App^{+/-}), and NPC1/APP double knockout (Npc1^{-/-}/App^{-/-}) mice were analyzed simultaneously.

Microarray Hybridization. For the microarray hybridization, the cerebella of pre-symptomatic wild-type (Npc1^{+/+}) and Npc1 knockout (Npc1^{-/-}) mice between 3 to 6-week of age were sent to GenUs (GenUs Biosystems, Northbrook, Ill.) for RNA processing and microarray hybridization. Briefly, RNA was extracted and purified using RiboPure® (Thermo Fisher Scientific, Waltham Mass.) according to manufacturer's instructions. Total RNA was quantitated by UV spectrophotometry (OD260/280), quality tested using Agilent Bioanalyzer, and prepared into cDNA. For microarray hybridization, the cRNA target was prepared from the DNA template and cRNA was fragmented to uniform size and hybridized to Agilent Mouse v2 GE 4×44 arrays. A separate v2 GE 4×44 microarray chip was used for each individual cerebellum sample, for a total of 6 chips (n=3 each for Npc1^{+/+} cerebella and Npc1^{-/-} cerebella). Slides were washed and scanned on the Agilent G2567 Microarray scanner and raw intensity values were normalized to the 75^{th} percentile intensity of each array using Agilent Feature Extraction and GeneSpring GX v7.3.1 software packages.

Transcriptome Analysis. Normalized raw expression data was first imported into R-software for transcriptome library generation and statistical analysis. Utilizing the standard R statistics code packages, the statistical significance of each transcript was calculated by the two-tailed student's t-test and the geometric means of each genotypes were used for fold-change (FC) calculation. Differentially expressed genes (DEGs) were selected by a combined cut-off for both fold-change (absolute FC>1.5) and p-value (p<0.05) as previously described (45). Next, the transcriptome data was imported into the Gene-set enrichment analysis software (GSEA, Broad Institute). For enrichment analysis, Hallmark database of the Molecular Signature Databases (MSigDB, Broad Institute) was used to identify significantly enriched gene-sets based on Normalized enrichment score (NES), false discovery rate (FDR), and nominal p-values calculated by the GSEA software. For molecular mapping and analysis, we utilized the Ingenuity Pathway Analysis software (IPA, Qiagen, Redwood City Calif.). Briefly, the comparative cerebellar transcriptome library of Npc1^{+/+} and Npc1^{-/-} were imported into IPA for core analysis which includes the upstream regulator analysis, causal network analysis, and disease and function analysis. Analysis cut-off was set for minimum absolute fold change >1.5 and p-value<0.05. P-value overlap and activation Z-score for molecular prediction analysis were calculated within the IPA software.

Immunocytochemistry. Mice brains of the indicated ages of Npc1^{-/-}/App^{+/+}, Npc1^{+/+}/App^{-/-}, Npc1^{-/-}/App^{+/+}, and Npc1^{-/-}/App^{-/-} genotypes were processed for immunohistochemistry. Sections 25 μm thick were cut sagittally through the cerebellum and mounted onto gelatin-chrome alum-coated Superfrost microscope slides (VWR, Denver, USA). Slides were placed on a warming surface at 37° C. for 30 minutes and rinsed with PBS for 10 minutes six times. Slides were incubated in blocking solution (PBS with 5% normal goat serum, 1% bovine serum albumin and 0.2% of 10% Triton x100) for 2 hours at room temperature. This step was followed by a 4° C. overnight incubation with CD3 antibody at 1:200 (Abcam 135372); incubation buffer consisted of PBS with 2% normal goat serum, 1% bovine serum albumin, and 0.1% Triton X-100. Following 3 washes in PBS with 0.1% Tween-20, slides were incubated in the dark with donkey anti-rabbit 488 secondary antibody (Abcam 21206) for 2 hours at room temperature; incubation buffer consisted of PBS with 2% normal goat serum, 1% bovine serum albumin and 0.1% Triton X-100. Samples were washed twice in PBS with 0.1% Tween-20 and once with PBS. Slides were mounted in Vectashield/DAPI hard-set mounting medium (Vectashield H-1500).

Controlled Cortical Impact Model. A controlled cortical impact model was used as a positive control for the presence of infiltrated T lymphocytes. Mice were anesthetized with isoflurane (1-3%), shaved, and surgical area cleaned with surgical soap, isopropyl alcohol and butadiene. A lidocaine injection was given prior to incision to expose the skull. After skin was retracted, a 5.0 mm diameter craniectomy-centered between bregma and lambda and 2.5 mm lateral to the sagittal suture—was performed to expose underlying dura and cortex. The injury was induced with a 3.0 mm flat-tipped, metal impactor. The impactor was centered within the craniectomy site and impact occurred with a velocity of 5.3 m/s, depth of 1.5 mm, and dwell time of 100 ms. Immediately following injury, the injury site was cleaned of blood and a polystyrene skull-cap was placed over the craniectomy site and sealed with VetBond. The incision was sutured and mice received an injection of saline for hydration and buprenorphine for pain prevention. Mice were placed in a heated recovery chamber and monitored for 1 hour prior to returning to home cage. Daily weights were taken for the first 7 days to monitor recovery. Injury parameters resulted in a moderately severe injury composed of cortical loss without overt hippocampal loss and sustained behavioral deficits. Tissue processing and CD3+ cell evaluation was carried out on 25 μm frozen cortical sections cut between bregma −3.5 and 1.0 to capture the lesion.

### Example 2: Multiplex Protein Analysis of NPC Cerebella Highlights IFN-γ-Responsive Pro-Inflammatory Cytokines

The Npc1^{-/-} cerebellar transcriptome results showed robust changes in innate immune genes, including various inflammatory cytokines and cytokine receptors (Table 1), congruent with previous reports of increased mRNA levels of inflammatory cytokines in NPC.

TABLE 1
Cytokines and cytokine receptors among the significant
DEGs in the pre-symptomatic Npc1^{-/-}cerebella.

| Genes: | FC | p-value |
|---|---|---|
| Mcp-1/Ccl2a | 3.286 | 0.026 |
| Rantes/Ccl5a | 4.772 | 0.0152 |
| Ccl6 | 4.237 | 0.0134 |
| Mcp3/Ccl7 | 1.961 | 0.0299 |
| Gcp-2/Cxcl6 | 1.754 | 0.0413 |
| Ip-10/Cxcl10a | 11.722 | 0.017 |
| Gmcsfrb/Csf2rb | 1.717 | 0.0134 |
| Il15ra | 1.772 | 0.0423 |
| Tgfb1 | 1.88 | 0.0074 |
| Ccl24 | −3.755 | 0.035 |
| Ccr10 | −1.904 | 0.007 |
| TnfrsfP | −1.641 | 0.0478 |
| Il17rd | −1.592 | 0.0387 |

DEGs selected by both FC and p-value cutoffs (absolute FC>1.5 and p<0.05).

However, in addition to the identification of individual innate immune genes, our systematic pathway analyses revealed that a novel and atypical activation pattern of IFN-γ- and IFN-α-responsive DEGs drive the four major inflammatory pathways identified in the Npc1^{-/-} cerebella (FIGS. 1-4). The protein levels of IFN-responsive pro-inflammatory cytokines in the pre-symptomatic Npc1^{-/-} cerebella (3 weeks), as well as the changes in their protein levels as neurodegeneration progresses to the terminal stage (12 weeks) were examined. Specifically, three prominent IFN-γ-responsive cytokines (Table 1) and nine cytokines predicted as potential master regulators by IPA upstream analysis were selected for validation. The levels of 20 additional prominent inflammatory cytokines were examined.

The cytokine analysis of 3-week old Npc1^{-/-} mouse cerebella revealed that IP10/CXCL10 is significantly upregulated in the early and pre-symptomatic stage NPC cerebella (FIG. 1A). Furthermore, the results showed that the increased expression of IP10/CXCL10 is exacerbated in the terminal stage (FIG. 1A).

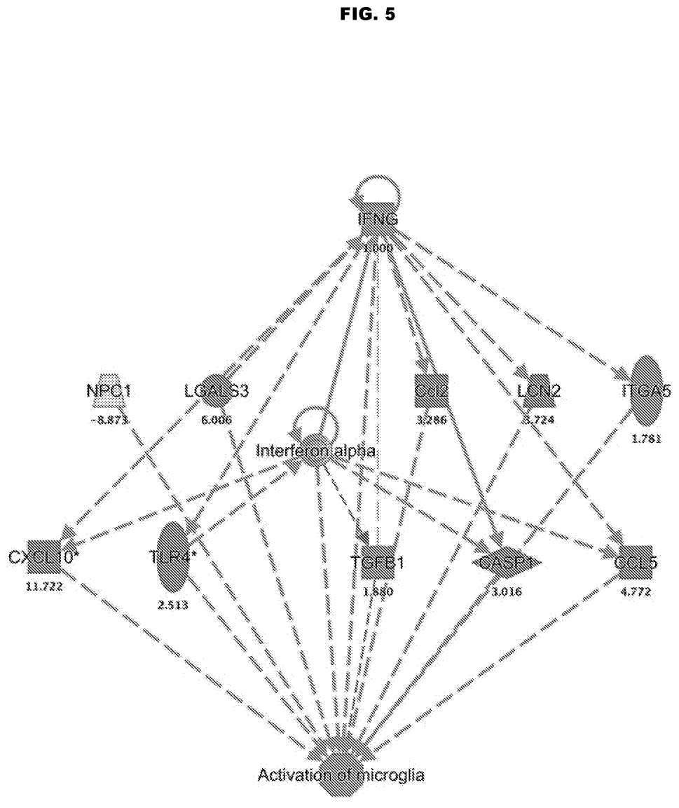

Of the 32 cytokines measured by the multiplex assay, at 3 weeks, interferon-gamma-induced protein 10 (IP-10/CXCL10) was the only molecule detected to be significantly elevated in the Npc1^{-/-} cerebella (FIG. 1A; compare 3 weeks wt (Npc1^{+/+}) with 3 weeks Npc1^{-/-}). Functionally, IP-10/CXCL10 is a potent downstream effector of IFN-γ and IFN-α, and is involved in all four major functional pathways identified in this study (FIG. 5). In the terminal stage Npc1^{-/-} cerebella, IP-10/CXCL10 was exacerbated compared to the pre-symptomatic stage (FIG. 1A; compare 3 weeks Npc1^{-/-} with 12 weeks Npc1^{-/-}) while no changes were observed in the wild-type animals.

The temporal progression of cytokine expression throughout the disease course of NPC was characterized by examining the levels of 32 pro- and anti-inflammatory cytokines at two distinct time points, at 3 and 12 weeks of age, representing the pre-symptomatic and the terminal-stage of the disease, respectively. The results showed that at 3 weeks, interferon-gamma induced protein 10 (IP10/CXCL10) was the only molecule significantly elevated in the Npc1^{-/-} cerebella, compared with the cerebella of the WT control littermates (FIG. 1A; compare WT3 with NPC3). Functionally, IP10/CXCL10 is a potent downstream effector of IFN-γ, the master regulator of the adaptive immune activation that is crucial in the transition from the innate immune response to the antigen-specific adaptive immune response. Therefore, the significant expression of IFN-γ responsive IP10/CXCL10 in 3-week old Npc1^{-/-} cerebella suggests that IFN-γ downstream signaling may be activated early in the neurodegenerative cascade of NPC. In the terminal stage Npc1^{-/-} cerebella, IP10/CXCL10 levels remained significantly increased compared with age-matched wild-type (Npc1^{+/+}) littermates (FIG. 1A; compare WT12 with NPC12). Additionally, the terminal stage cerebella also displayed significantly elevated levels of monokine induced by gamma interferon (MIG/CXCL9), monocyte chemoattractant protein-1 (MCP-1/CCL2), macrophage inflammatory protein-1-alpha (MIP-1α/CCL3), macrophage inflammatory protein-1-beta (MIP-1β/CCL4), regulated on activation normal T cell expressed and secreted (RANTES/CCL5), interleukin-1-alpha (IL-1α), Eotaxin (CCL11), and Keratinocyte Chemoattractant (KC/CXCL1) (FIG. 1B-1I). Previously, IP10/CXCL10, MIG/CXCL9, MCP-1/CCL2, MIP-1α/CCL3, MIP-1β/CCL4, and RANTES/CCL5 have been shown to be upregulated in response to IFN-γ (27, 30, 31). Therefore, the cytokine profile of the terminal stage, displaying the increased expression of several IFN-γ-responsive cytokines, suggests that the early activation of IFN-γ downstream signaling remains sustained throughout the disease course in Npc1^{-/-} mouse cerebella.

In the terminal stage Npc1^{-/-} cerebella, eight IFN-γ- and/or IFN-α-responsive cytokines were elevated, including monokine induced by gamma interferon (MIG/CXCL9) (FIG. 1B), monocyte chemoattractant protein-1 (MCP-1/CCL2) (FIG. 1C), macrophage inflammatory protein-1-alpha (MIP-1α/CCL3) (FIG. 1D), macrophage inflammatory protein-1-beta (MIP-1β/CCL4) (FIG. 1E), regulated on activation normal T cell expressed and secreted (RANTES/CCL5) (FIG. 1F), macrophage colony-stimulating factor (M-CSF) (FIG. 1G), interleukin-1-alpha (IL-1α) (FIG. 1H), and keratinocyte chemoattractant (KC/CXCL1) (FIG. 1I).

Additionally, Interleukin-15 (IL-15) was reduced (FIG. 1J) and eotaxin (CCL11) (FIG. 1K) and leukemia inhibitory factor (LIF) (FIG. 1L) were elevated in terminal stage Npc1^{-/-} cerebella.

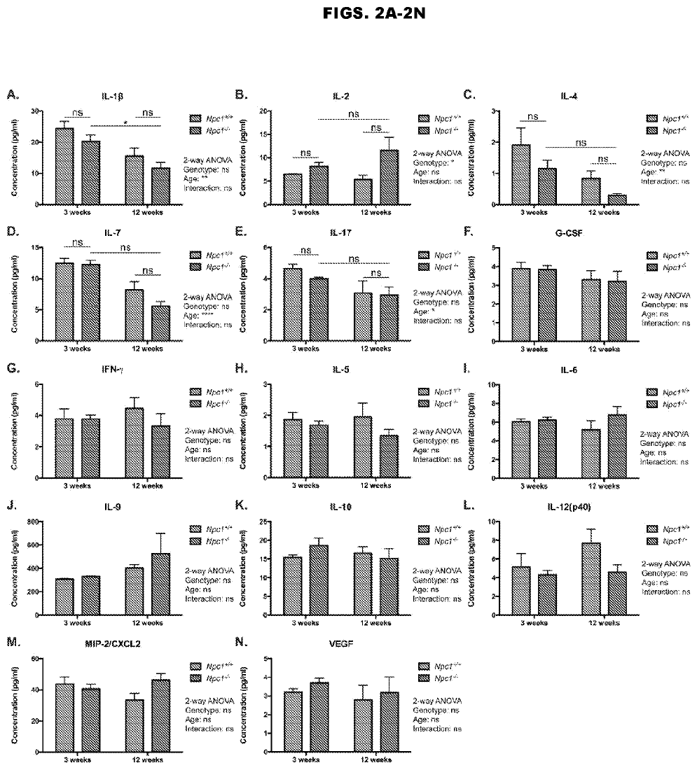

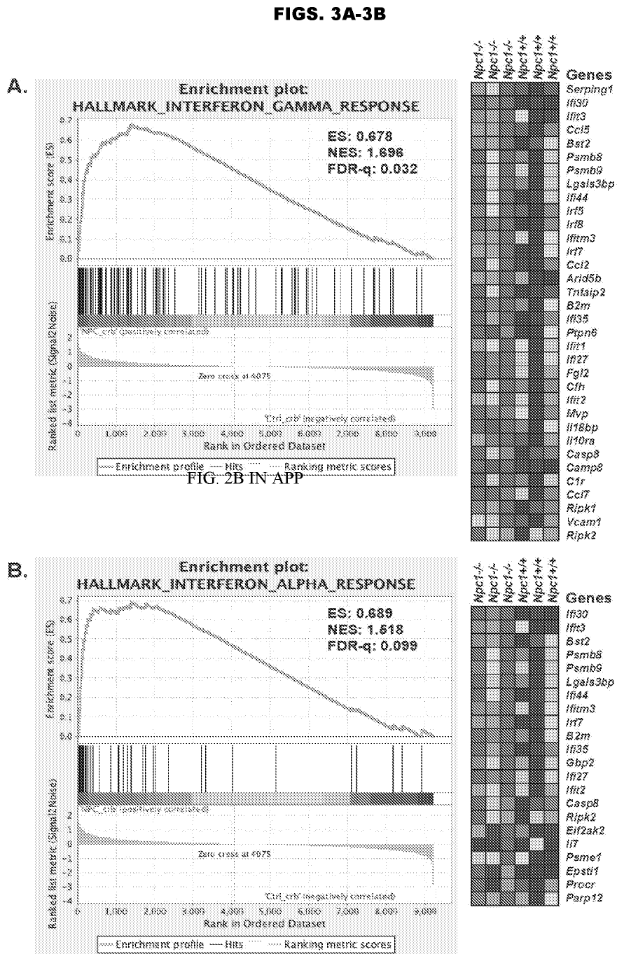

Lastly, fourteen cytokines showed no significant differences between genotypes at either time points-IL-1β, as shown in FIG. 2A; IL-2, as shown in FIG. 2B; IL-4, as shown in FIG. 2C; IL-7, as shown in FIG. 2D; IL-17, as shown in FIG. 2E; G-CSF, as shown in FIG. 2F; IFN-γ, as shown in FIG. 2G; IL-5, as shown in FIG. 211; IL-6, as shown in FIG. 21; IL-9, as shown in FIG. 2J; IL-10, as shown in FIG. 2K; IL-12 (p40), as shown in FIG. 2L; MIP-2/CXCL2, as shown in FIG. 2M; and VEGF, as shown in FIG. 2N. Six cytokines (GM-CSF, IL-3, IL-12(p70), IL-13, LIX/CXCL5, and TNF-α) were below the detectable range of the multiplex assay.

### Example 3: Genome-Wide Transcriptome Analysis of Pre-Symptomatic NPC Cerebella Confirms the Early Activation of Genes Downstream of IFN-γ and IFN-α

Functionally, IP10/CXCL10 is a crucial downstream effector of the IFN-γ system through the chemotaxis of CXCR3+ immune cells, particularly CD4+ and CD8+T-lymphocytes, to the site of CNS inflammation. In addition, IP10/CXCL10 also plays a major role in the development and antigen-specific activation of T-lymphocytes. Accordingly, the robust activation of IP10/CXCL10 suggests that early signaling associated with T-lymphocyte activation and recruitment may be detectable in pre-symptomatic Npc1^{-/-} mouse cerebella.

A genome-wide transcriptome analysis was utilized to further elucidate the potential activation of IFN-γ downstream signaling in pre-symptomatic NPC mouse brain. Cerebellar transcriptome was generated from pre-symptomatic Npc1^{-/-} mice and WT littermates and microarray hybridization technique yielded 39,429 transcript reads from which the differentially expressed genes (DEGs) were selected. In total, 387 DEGs were identified in the Npc1^{-/-} cerebella compared to the wild-type controls, of which 176 genes were upregulated and 211 genes were downregulated. The Npc1^{-/-} cerebellar transcriptome was analyzed utilizing the Gene-Set Enrichment Analysis (GSEA, Broad Institute), a software that effectively identifies majorly affected pathways within large-omics data by analyzing the enrichment of gene groups by function or location. GSEA results revealed that the IFN-γ downstream genes were indeed robustly upregulated in the pre-symptomatic Npc1^{-/-} cerebella. FIGS. 3A and 3B show the expression of genes in the Npc1^{-/-} cerebellar transcriptome utilizing the Gene-Set Enrichment Analysis (GSEA). The Interferon Gamma Response gene-set within the Hallmark database of the Molecular Signature Databases (MSigDB, Broad Institute) was the most enriched gene-set with normalized enrichment score of 1.695, nominal p-value of 0.000, and false discovery rate q-value of 0.032 (FIG. 3A). Interestingly, GSEA revealed that genes downstream of IFN-α signaling were also upregulated the pre-symptomatic Npc1^{-/-} cerebella. The Interferon Alpha Response gene-set was shown to be significantly enriched with normalized enrichment score of 1.519, nominal p-value of 0.000, and false discovery rate q-value of 0.099 (FIG. 3B).

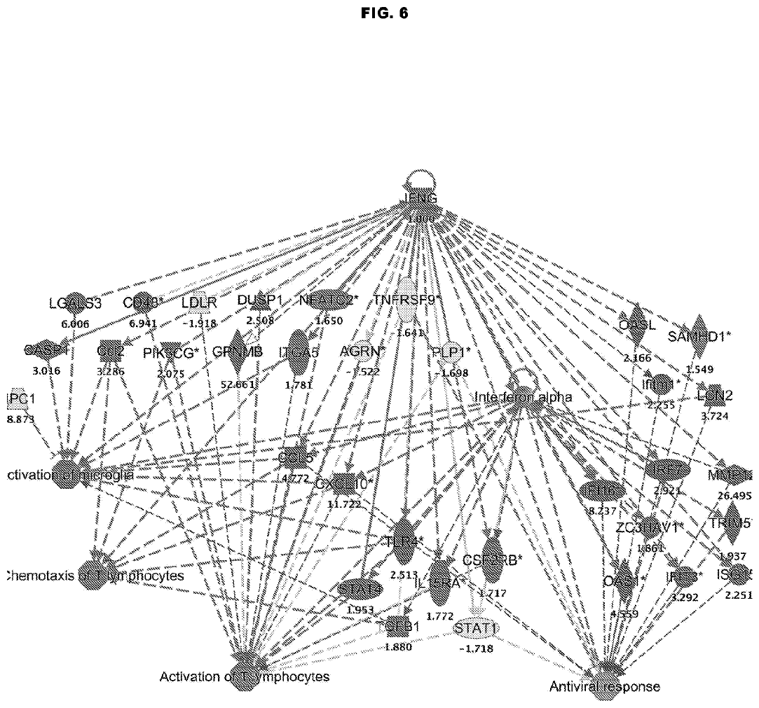

Next, the Ingenuity Pathway Analysis software (IPA, Qiagen, Redwood City Calif.) was utilized to further map out the molecular functions and relationships of differentially expressed interferon-responsive genes identified within the Npc1^{-/-} cerebellar transcriptome. FIG. 4A shows the mapping of the molecular functions and relationships of differentially expressed interferon-responsive genes identified within the Npc1^{-/-} cerebellar transcriptome using the Ingenuity Pathway Analysis software (IPA, Qiagen). Red indicates upregulation and green indicates downregulation. DEGs plotted in their respective sub-cellular location; p<0.05 with each FC-value listed below the gene symbol. *Duplicate identifiers used for the same gene. FIG. 4B presents the IPA key for molecule shape, color, and interaction. Consistent with the GSEA findings, IPA results again highlighted that IFN-γ is the most likely upstream master regulator of the DEGs identified in the pre-symptomatic Npc1^{-/-} cerebellar transcriptome, based on both the p-value overlap ranking (IFN-γ, p=4.17 E-14) and the z-score ranking (IFN-γ, Z=4.533). Systematic IPA causal network analysis revealed that IFN-γ activation is likely to be upstream of 60 DEGs identified in the pre-symptomatic Npc1^{-/-} cerebella (FIGS. 4A and 4B), as well as 18 other predicted upstream regulators of the entire transcriptome. The genome-wide transcriptome analysis of pre-symptomatic NPC cerebella showed robust upregulation of IP10/Cxcl10 (11.722 fold up, p<0.05), as well as 59 other IFN-γ-responsive genes (FIGS. 4A and 4B). Altogether, 48 IFN-γ-responsive genes were upregulated and 12 genes were downregulated (FIGS. 4A and 4B). In addition, IPA results highlighted that IFN-α is also among the top predicted upstream regulator with 23 DEGs linked directly as IFN-α downstream genes (FIGS. 4A and 4B). Furthermore, IPA disease and function analysis confirmed that the IFN-γ-responsive DEGs identified in pre-symptomatic NPC cerebella are involved in T-lymphocyte activation and chemotaxis (FIGS. 4A, 4B, and 6).

Next, the functional roles of the IFN-γ-responsive genes identified in the early pathologic state of the NPC cerebellar degeneration were assessed. IPA disease and function analysis identified that nine IFN-γ-responsive genes directly related to the activation of microglia are differentially expressed in the early NPC cerebella, including: Lgals3, Mcp1/Ccl2, Lcn2, Itga5, IP10/Cxcl10, Tlr4, Tgfb1, Casp1, and Rantes/Ccl5 (FIG. 5). Additionally, IP10/Cxcl10, Tlr4, Tgfb1, Casp1, and Rantes/Ccl5 involved in microglial activation have also been shown to be downstream of IFN-α activation through IPA upstream analysis (FIG. 5).

Next, the IPA results revealed that in the absence of an active infection, 15 IFN-γ-responsive genes involved in anti-viral response are upregulated in the pre-symptomatic Npc1^{-/-} cerebella, including Tnfrsf9, Plp1, IP10/Cxcl10, Rantes/Ccl5, Oasl, Stat1, Samhd1, Lcn2, Ifitm1, Ifi16, Ifit3, Mmp12, Isg15, Irf7, and Oas1 (FIG. 5). Of these, IPA identified that 9 of the 15 genes were also linked to predicted IFN-α activation (FIG. 5: IP10/Cxcl10, Rantes/Ccl5, Stat1, Ifi16, Ifit3, Mmp12, Isg15, Irf7, and Oas1). Furthermore, IPA identified two additional IFN-α family downstream anti-viral genes Trim5 and Zc3hav1 (FIG. 4).

FIG. 6 shows the merged network of IFN-γ- and IFN-α-responsive DEGs involved in microglial activation, anti-viral response, activation of T-lymphocytes, and chemotaxis of T-lymphocytes. The IPA functional analysis revealed that genes related to T-lymphocytes were significantly enriched in the pre-symptomatic Npc1^{-/-} cerebella. IPA showed 18 IFN-γ-responsive genes involved in T-lymphocyte activation were differentially expressed, including Mcp1/Ccl2, Pik3cg, Cd48, Ldlr, Gpnmb, Nfatc2, Dusp1, Itga5, Agrn, Tnfrsf9, Plp1, IP10/Cxcl10, Rantes/Ccl5, Tgfb1, Il15ra, Stat1, Tlr4, and Csf2rb (FIG. 6). Seven of these 18 IFN-γ-responsive genes were also shown to be linked to IFN-α activation (FIG. 6: IP10/Cxcl10, Rantes/Ccl5, Tgfb1, Il15ra, Stat1, Tlr4, and Csf2rb). In addition, IPA identified that six IFN-γ-responsive genes are also involved in chemotaxis of activated T-lymphocyte (FIG. 5: Mcp1/Ccl2, Pik3cg, IP10/Cxcl10, Rantes/Ccl5, Tgfb1, and Tlr4). Four of the genes involved in T-lymphocyte chemotaxis were also linked to the predicted activation of IFN-α (FIG. 5: IP10/Cxcl10, Rantes/Ccl5, Tgfb1, and Tlr4).

The genome-wide transcriptome analysis revealed the upregulation of genes involved in microglial activation and anti-viral response (FIGS. 5 and 6). The finding that IFN-γ-responsive genes related to microglial activation are upregulated in pre-symptomatic NPC animals is of particular importance, because microglia activation is prominent in the NPC brain, and the analysis indicates that the IFN-γ system- and particularly the early activation of IP10/CXCL10—may be a key early mediator of this pathology (FIGS. 1A and 4). Similarly, the activation of IFN-γ-responsive anti-viral genes in pre-symptomatic NPC cerebellum is also of interest, given the newly discovered link between NPC1 and viral infection. NPC1 is involved in the pathogenesis of viral infection and IFN-γ is crucial in the adaptive immune signaling, a crucial mechanism in anti-viral response. In addition, NPC1 is also implicated in the host infection of the intracellular bacterial pathogen, Mycobacterium tuberculosis. In both viral and mycobacterial infections, the IFN-γ system plays a crucial role in activating the adaptive immune response against intracellular pathogens and defects in IFN-γ signaling results in refractory viral and mycobacterial infections. Here, it is interesting to note that defect in IFN-γ-downstream IP10/CXCL10 also results in vulnerability to viral and bacterial infections, thereby highlighting the significant functional role of IP10/CXCL10 in the IFN-γ signaling cascade in relation to anti-microbial function. Taken together, the activation of IFN-γ-responsive anti-viral genes in the pre-symptomatic cerebella of NPC, in the absence of pathogenic infection, suggests that NPC1 defect aberrantly triggers various cellular defense mechanisms intended for intracellular pathogens.

Further, it is also important to note that many of the IFN-γ-responsive genes involved in the four-major functional pathway are also linked to the predicted IFN-α activation (FIGS. 5 and 6). While IFN-γ and IFN-α downstream pathways are often considered separate, there is overlap of IFN-γ and IFN-α functions. For example, both IFN-γ and IFN-α induce IP10/CXCL10, a key T-lymphocyte chemokine and a major inflammatory marker of the pre-symptomatic NPC brain (FIG. 1A).

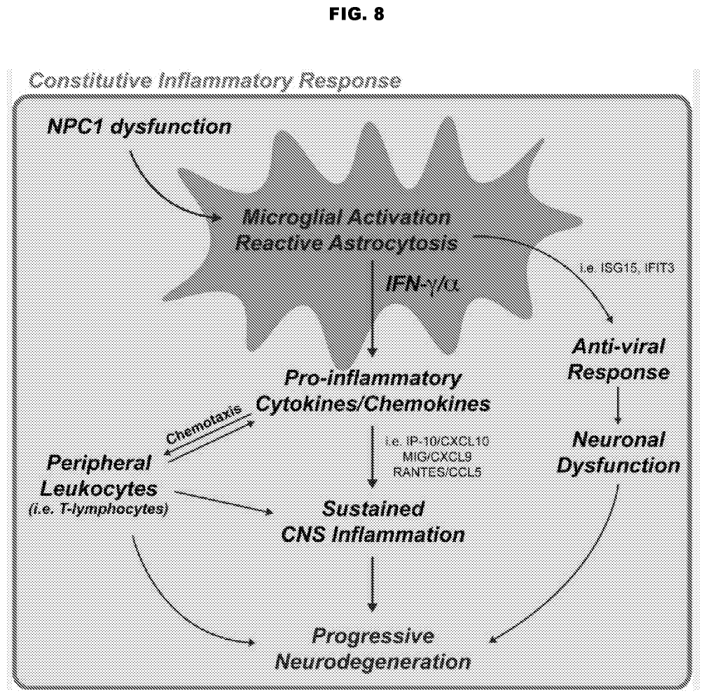

FIG. 8 is a schematic representation of the mechanism of NPC neuroinflammation. Dysfunction of NPC1 protein results in the aberrant activation of microglia and astrocytes in the CNS milieu. Subsequently, the constitutive pro-inflammatory response driven by IFN-γ and IFN-α downstream signaling result in the secretion of pro-inflammatory cytokines and chemokines (i.e. IP-10/CXCL10, MIG/CXCL9, RANTES/CCL5) which sustains the chronic neuroinflammation and mechanistically contribute to the progressive neurodegeneration observed in NPC pathology. Chemotaxis of peripheral leukocytes (i.e. activated T-lymphocytes) results in additional cytokine/chemokine production and further exacerbation of CNS inflammation. Sustained inflammation, including the induction of anti-viral state and anti-viral proteins (i.e. ISG15 and IFIT3), exacerbates the neuronal dysfunction observed in NPC and contribute to neurodegeneration.

### Example 4—Genome-Wide Transcriptome Analysis of Pre-Symptomatic Cerebella Reveals that Loss of APP Exacerbates the Early Activation of Aberrant IFN-γ Downstream Signaling in NPC Mice

Genome-wide transcriptome analysis of pre-symptomatic cerebella reveals that loss of APP exacerbates the early activation of aberrant IFN-γ downstream signaling in NPC mice. Microarray hybridization yielded 39,429 transcript reads from which differentially expressed genes (DEGs) were selected by combining a fold-change cutoff (absolute change >1.5) and a p-value cutoff (p<0.05). From Npc1^{-/-}/App^{-/-} cerebella, 6,269 transcript-reads (TRs) displayed an absolute fold-change (aFC) greater than 1.5 (FC<−1.5 or FC>1.5) and 1,534 TRs were statistically significant (p<0.05) compared with the wild-type (Npc1^{+/+}/App^{+/+}), analyzed by one-way ANOVA and Tukey's post hoc test (Table 2). In total, 891 DEGs were identified (following transcript ID to gene mapping), of which 418 genes were upregulated and 473 genes were downregulated. In Npc1^{-/-}/App^{+/+} samples, 3,967 TRs displayed aFC>1.5 and 684 TRs were statistically significant (p<0.05). In total, 431 DEGs were identified (following transcript ID to gene mapping), of which 252 genes were upregulated and 179 genes were downregulated (Table 2). In Npc1^{-/-}/App^{-/-} cerebella, 7,132 TRs displayed aFC>1.5 and 3,359 TRs were statistically significant (p<0.05). In total, 1,973 DEGs were identified (following transcript ID to gene mapping), of which 1,265 genes were upregulated and 708 genes were downregulated (Table 2).

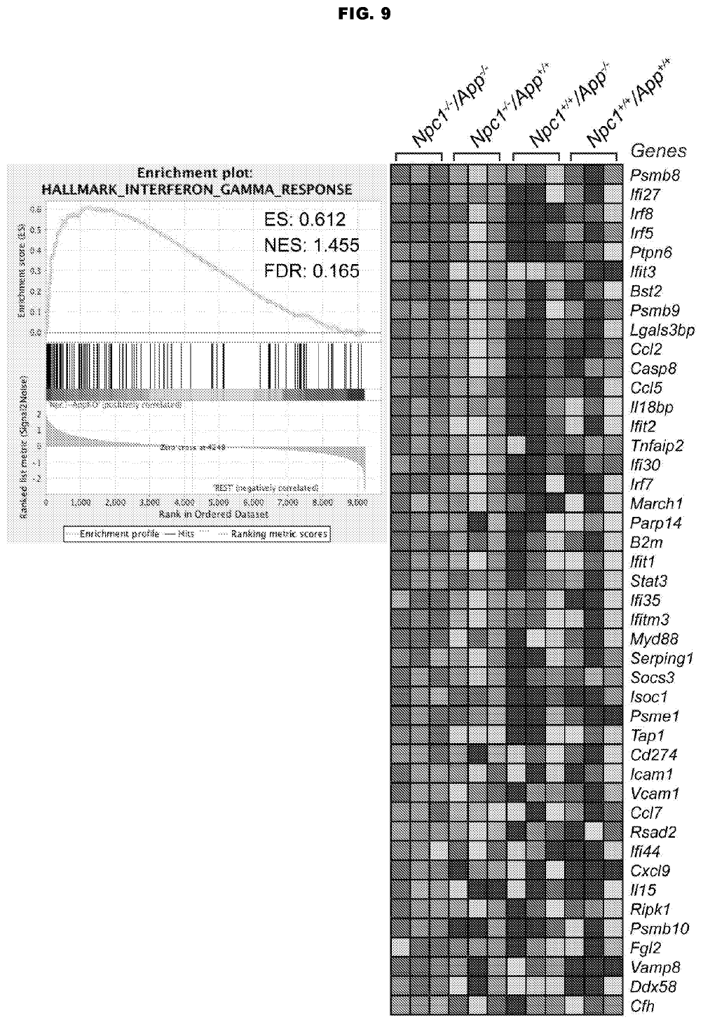

Comparative analyses of wild-type cerebella vs. Npc1^{-/-}/App^{-/-}, Npc1^{-/-}/App^{+/+}, and Npc1^{-/-}/App^{-/-} revealed that the loss of APP results in a significant exacerbation of the aberrant IFN-γ downstream signaling previously characterized in pre-symptomatic Npc1^{-/-} /App^{+/+} mice. Gene set enrichment analysis (GSEA) revealed that Interferon Gamma Signaling gene set was significantly enriched in the Npc1^{-/-}/App^{-/-} mouse cerebellar transcriptome (NES=1.455 and FDR=0.165), in comparison to Npc1^{+/+}/App^{+/+}, Npc1^{+/+}/App^{-/-}, and Npc1^{-/-}/App^{+/+}. FIG. 9 is a GSEA that reveals the activation of Interferon Gamma Response gene sets in Npc1^{-/-}/App^{-/-} mouse cerebella compared with the three remaining genotypes (Npc1−/−/App−/− vs. remaining genotypes). ES=enrichment score, NES=normalized enrichment score, FDR-q=false discovery rate q-value.

Ingenuity Pathway Analysis confirmed that Npc1^{-/-}/App^{-/-} mouse cerebellar transcriptome indeed displayed a significant increase in IFN-γ-responsive genes (FIG. 10A). Compared with a single knockout mouse model of NPC (Npc1^{-/-}/App^{+/+}) which displayed aberrant differential expression of 60 IFN-γ-responsive genes in the pre-symptomatic stage, Npc1^{-/-}/App^{-/-} mouse cerebella displayed the differential expression of 262 IFN-γ-responsive genes (FIG. 10A). Of those, 223 were upregulated and 39 were downregulated. In addition, IPA Upstream Analysis revealed that IFN-γ is the most likely upstream master regulator of 1,973 DEGs identified in the Npc1^{-/-}/App^{-/-} mouse cerebellar transcriptome (Table 3). This finding is congruent with our previous report that IFN-γ is the top master regulator of 387 DEGs identified in the Npc1^{-/-} cerebellar transcriptome.

TABLE 2
Differentially expressed genes identified in each genotype
by genome-wide transcriptome analysis. TR = number
of transcript-reads by microarray. aFC = absolute
fold-change. DEG = Differentially expressed gene
(mapped ID + statistically significant by FC and p cutoffs).

| | Npc1^{+/+}/ | Npc1^{-/-}/ | Npc1^{-/-}/ |
|---|---|---|---|
| | App^{-/-} | App^{+/+} | App^{-/-} |
| TR (aFC > 1.5) | 6,269 | 3,967 | 7,132 |
| TR (p < 0.05) | 1,534 | 684 | 3,359 |
| DEG (FC + p) | 891 | 431 | 1,973 |
| DEG (up) | 418 | 252 | 1,265 |
| DEG (down) | 473 | 179 | 708 |

TABLE 3
Top 8 predicted cytokine/chemokine upstream regulators of DEGs identified in
Npc1_{-/-}/App_{-/-}, Npc1_{-/-}/App_{+/+}, and Npc1_{+/+}/App_{-/-} mouse cerebella.
IPA Upstream Analysis and Comparison Analysis identified eight cytokines and chemokines
upstream master regulators in each genotype, compared with the wild-type (Npc1_{+/+}/App_{+/+})
littermates. Each of the three columns (Z-score, −log(p), and # T.M.) across the
three genotypes are heatmaps. Red = enriched, Green = down, and White = zero.
Z-scores and p-values calculated by IPA software. #T.M. = number of downstream
target molecules; WT = wild-type.

| | Npc1^{-/-}/App^{-/-} vs. WT | | | Npc1^{-/-}/App^{+/+} vs. WT | | | Npc1^{+/+}/App^{-/-} vs. WT | | |
|---|---|---|---|---|---|---|---|---|---|
| Upstream Regulator | Z-score | −log(p) | #T.M. | Z-score | −log(p) | #T.M. | Z-score | −log(p) | #T.M. |
| IFN-γ | 9.324 | 38.497 | 262 | 5.432 | 21.225 | 84 | −0.152 | 2.481 | 69 |
| TNFα | 6.724 | 17.390 | 258 | 4.694 | 12.412 | 81 | −0.816 | 1.324 | 79 |
| IFN-α (group) | 6.567 | 14.712 | 84 | 2.981 | 11.699 | 33 | −1.845 | 0 | 12 |
| GM-CSF/CSF2 | 5.761 | 8.539 | 79 | 4.023 | 5.740 | 26 | −0.239 | 2.119 | 28 |
| IFN-β1 | 4.841 | 11.953 | 62 | 2.874 | 7.525 | 19 | 1.250 | 2.844 | 0 |
| IL-1β | 6.972 | 13.230 | 147 | 3.828 | 9.729 | 49 | n/a | 0 | n/a |
| IFN-α2 | 6.302 | 16.475 | 61 | 3.059 | 13.590 | 27 | n/a | 0 | n/a |
| IFN-β (group) | 4.956 | 7.919 | 31 | 3.595 | 10.769 | 18 | n/a | 0 | n/a |

### Example 5—Loss of APP Exacerbates the Early Activation of Aberrant IFN-α Downstream Signaling in NPC Mice

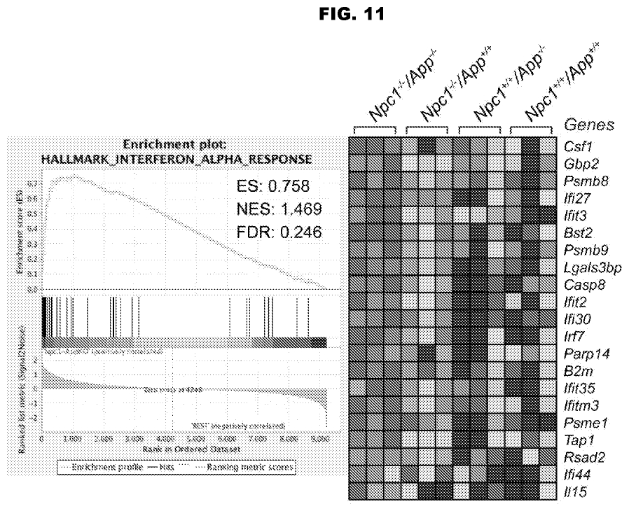

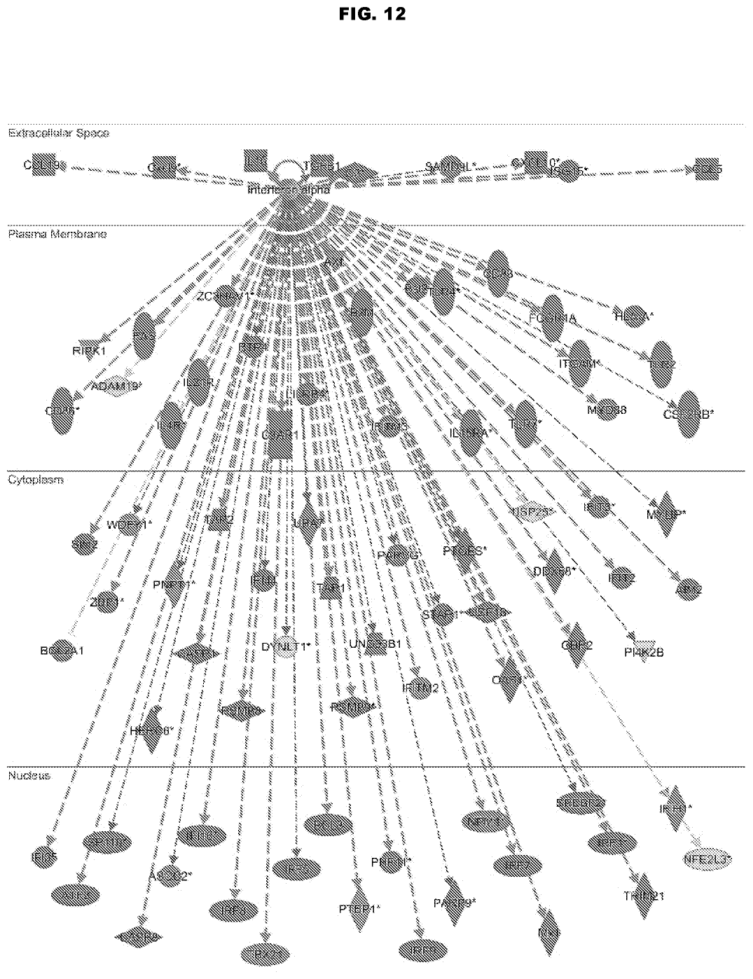

Comparative analyses of wild-type cerebella versus Npc1^{+/+}/App^{-/-}, Npc1^{-/-}/App^{+/+}, and Npc1^{-/-}/App^{-/-} also revealed that loss of APP exacerbates the aberrant IFN-α downstream signaling seen in pre-symptomatic Npc1^{-/-}/App^{+/+} mice. GSEA showed that the Interferon Alpha Signaling gene set is significantly enriched in the Npc1^{-/-}/App^{-/-} mouse cerebellar transcriptome (NES=1.469 and FDR=0.246), when compared with the Npc1^{-/-}/App^{+/+}, Npc1^{+/+}/App^{-/-}, and Npc1^{-/-}/App^{+/+} genotypes (FIG. 11). IPA further confirmed that 84 IFN-α-responsive genes are differentially expressed in Npc1^{-/-}/App^{-/-} mouse cerebella when compared with wild-type (Npc1^{+/+}/App^{+/+}) controls (FIG. 12). Of the 84 DEGs, 79 IFN-α-responsive genes were upregulated and 5 IFN-α-responsive genes were downregulated (FIG. 12). This is a substantial increase from the differential expression of 23 IFN-α-responsive genes in Npc1^{-/-} mice versus wild-type controls.

### Example 6—Loss of APP Results in the Exacerbation of NPC-Specific Inflammatory Pathways Mediated by IFN-γ- and IFN-α-Responsive Genes

There are four major inflammatory pathways that are aberrantly activated in pre-symptomatic Npc1^{-/-} mouse cerebella: activation of microglia, anti-viral response, and T-lymphocyte activation and chemotaxis. Here, in Npc1^{-/-}/App^{-/-} mice, the aberrant activation of all four NPC-specific inflammatory pathways was exacerbated: IPA Disease and Function Analysis revealed strong activation of microglia in the Npc1^{-/-}/App^{-/-} mouse cerebellum, as measured by the identification of 29 significant DEGs associated with this pathway (FIG. 15). Of these, 25 were IFN-γ-responsive genes and 7 were IFN-α-responsive, a substantial change from the 9 IFN-γ-responsive and 5 IFN-α-responsive genes related to microglial activation previously identified in the Npc1^{-/-} cerebellum. Antiviral response was also strongly activated in Npc1^{-/-}/App^{-/-} mouse cerebella, as revealed by the presence of 56 DEGs related to this pathway (FIG. 16), 47 of which were IFN-γ-responsive and 39 IFN-α-responsive, again representing a substantial increase compared with the 15 IFN-γ-responsive and 9 IFN-α-responsive altered genes previously identified in the Npc1^{-/-} cerebellum. Disease and Function Analysis and Upstream Analysis also identified 83 significantly DEGs related to antimicrobial response in the Npc1^{-/-}/App^{-/-} cerebella transcriptome compared with wild-type mice (Npc1^{+/+}/App^{+/+}; FIG. 17). Of those, 62 were IFN-γ-responsive genes and 44 were IFN-α-responsive genes. The DEGs involved in activation of antimicrobial response showed a significant overlap (56 genes) with the antiviral response (FIG. 16) but additional genes involved in antimicrobial immune response were also identified (FIG. 17).

Activation of T-lymphocytes was also present in Npc1^{-/-}/App^{-/-} cerebella, as evidence by the presence of 87 linked DEGs (FIG. 18). Of these, 77 were IFN-γ-responsive and 34 were IFN-α-responsive. Interestingly, IPA also showed that T-lymphocyte co-stimulatory ligand receptor CD28 was also implicated in the Npc1^{-/-}/App^{-/-} cerebellum (FIG. 19). CD28 is a T-lymphocyte co-receptor for membrane-bound-ligands on antigen-presenting cells, such as CD80 and CD86, that are required for T-lymphocyte activation and survival. In Npc1^{-/-}/App^{-/-} cerebella, 42 DEGs downstream of predicted CD28 activation were identified by IPA (FIG. 19), thereby providing additional insight into the potential mechanism by which APP loss of function may contribute to the IFN-mediated T-lymphocyte activation seen in the Npc1^{-/-}/App^{-/-} mouse cerebella (FIG. 18). IPA also showed that the aberrant expression of DEGs related to chemotaxis of T-lymphocytes in NPC is exacerbated by the loss of APP (FIG. 20). In Npc1^{-/-}/App^{-/-}, 25 DEGs related to chemotaxis of T-lymphocytes were identified, of which 18 were IFN-γ-responsive and 8 were IFN-α-responsive (FIG. 20). By comparison, 6 IFN-γ-responsive genes and 4 IFN-α-responsive genes were identified as related to chemotaxis of T-lymphocytes in Npc1^{-/-} cerebella.

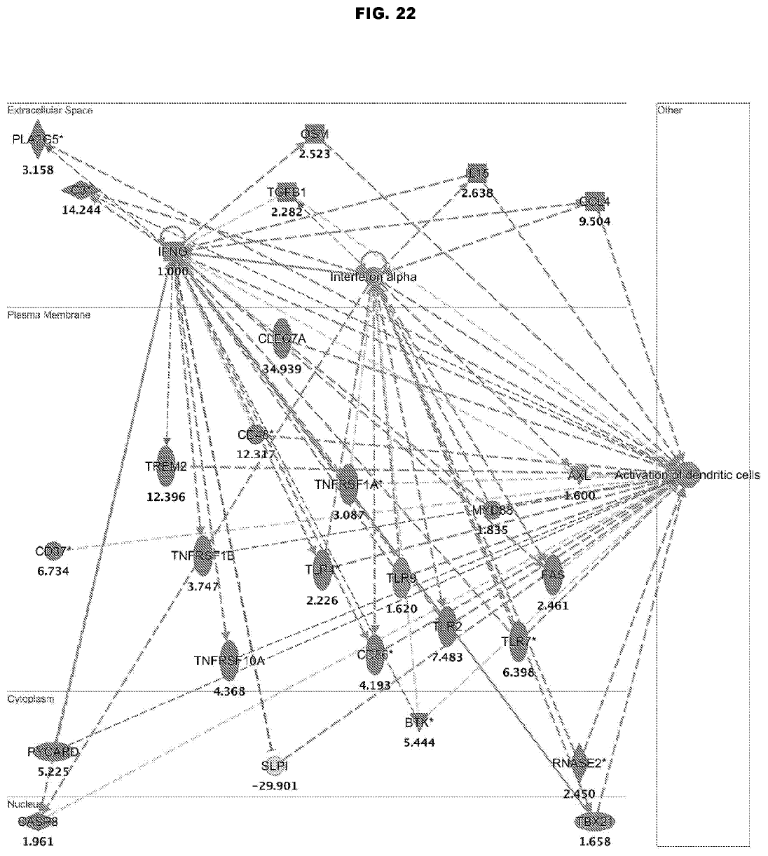

IPA Disease and Function Analysis identified 87 significantly DEGs related to the activation of antigen presenting cells (APCs) in Npc1^{-/-}/App^{-/-} mice, compared with wild-type controls (Npc1^{+/+}/App^{+/+}; FIG. 21). The combination of IPA Disease and Function Analysis and Upstream Analysis further identified 85 IFN-γ-responsive genes and 35 IFN-α-responsive genes related to antigen presentation in Npc1^{-/-}/App^{-/-} cerebella, highlighting antigen presentation as one of the main inflammatory mechanisms related to APP loss of function in the NPC brain (FIG. 21). More specifically, the activation of dendritic cells was implicated in Npc1^{-/-}/App^{-/-} mouse cerebella, as IPA Disease and Function Analysis unveiled 27 DEGs linked to this pathway (FIG. 22). All differentially expressed genes (DEGs) are localized to their sub-cellular location. All plotted DEGs meet the significance cutoff of fold-change (absolute FC>1.5) and p-value (p<0.05). *Duplicate identifiers used for the same gene. A detailed key for IPA molecular shape, color, and interaction is provided in FIG. 4B.

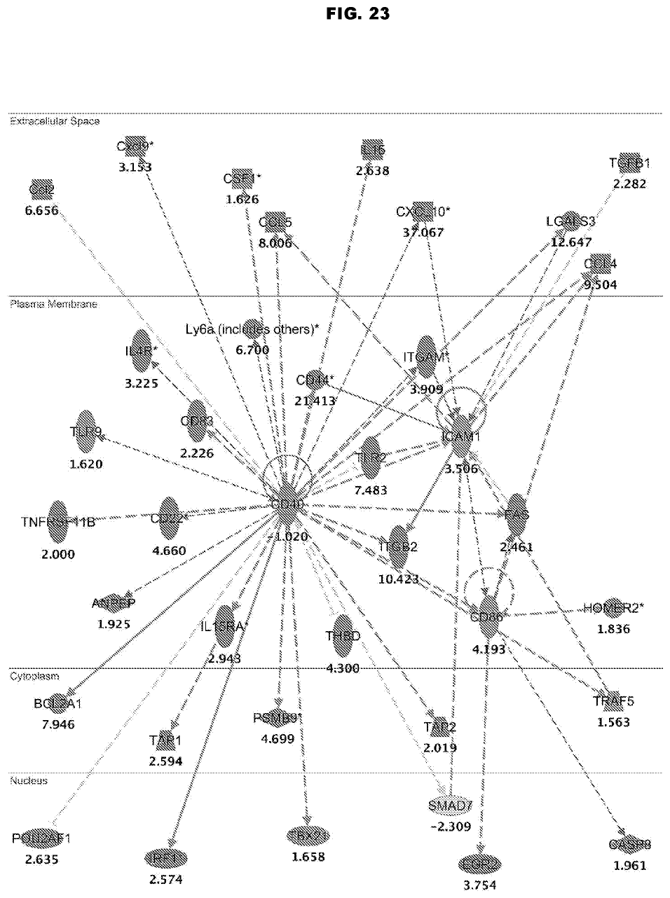

Of these, 25 were IFN-γ-responsive and 17 were IFN-α-responsive, further validating the notion of IFN exacerbation as a consequence of APP loss of function. Finally, IPA Upstream Analysis showed that genes downstream of the co-stimulatory molecules involved in APC-mediated activation of the adaptive immune system are significantly enriched in Npc1^{-/-}/App^{-/-} cerebella, with 32 DEGs mapping to CD40, 6 mapping to CD86, and 4 mapping to ICAM1 (FIG. 23). In Npc1−/−/App−/− mouse cerebella, 32 genes related to CD40, 12 genes related to ICAM1, and 6 genes related to CD86 were differentially expressed when compared with wild type littermates (Npc1+/+/App+/+). All differentially expressed genes (DEGs) are localized to their sub-cellular location. All plotted DEGs meet the significance cutoff of fold-change (absolute FC>1.5) and p-value (p<0.05). *Duplicate identifiers used for the same gene. A detailed key for IPA molecular shape, color, and interaction is provided in FIG. 4B.

### Example 7—Multiplex Protein Analysis Across Npc1 and App Genotypes: NPC Pre-Symptomatic Stage

In order to identify how the loss of each App allele affects the protein expression of pro- and anti-inflammatory cytokines (downstream of IFN signaling), we utilized a multiplex cytokine analysis to simultaneously determine the protein levels of 32 cytokines in the following genotypes: Npc1^{+/+}/App^{+/+}, Npc1^{+/+}/App^{-/-}, Npc1^{-/-}/App^{+/+}, Npc1^{-/-}/App^{+/-}, and Npc1^{-/-}/App^{-/-}. In 3-week-old cerebella across all five genotypes, 26 cytokines were expressed within detectable levels but only 5 of them displayed significant differential expression in either Npc1^{-/-}/App^{+/+}, Npc1^{-/-}/App^{+/-}, or Npc1^{-/-}/App^{-/-} compared with wild-type littermate control (FIGS. 13A-13F). IFN-γ downstream effector cytokine, IP-10/CXCL10, was the only cytokine significantly increased in Npc1^{-/-}/App^{+/+} at 3 weeks (FIG. 13A), and loss of a single App allele in the Npc1 brain (Npc1^{-/-}/App^{+/-}) was sufficient to trigger an additional increase in IP-10/CXCL10 expression.

In addition, one IFN-γ downstream cytokine, RANTES/CCL5, displayed an increased trend in 3-week old Npc1^{-/-}/App^{+/+} mice compared with wild-type littermates, but did not reach statistical significance (FIG. 13B). By contrast, loss of a single App allele in NPC mice (Npc1^{-/-}/App^{+/-}) was sufficient to significantly increase its expression (FIG. 13B). Eotaxin/CCL11 was increased in Npc1^{-/-}/App^{+/+}, but this increase did not reach statistical significance (FIG. 5C). Interestingly, loss of App in a wild-type background also showed an increased trend, but the impact of App loss on eotaxin expression is only significant in the NPC brain following loss of both App alleles (FIG. 13C). Expression of IL-10 was not significantly altered in single gene knockouts (Npc1^{+/+}/App^{-/-} and Npc1^{-/-}/App^{+/+}) at 3 weeks of age, compared with wild-type controls (FIG. 13D). However, in the NPC brain, loss of both App alleles (Npc1^{-/-}/App^{-/-}) resulted in a statistically significant increase in expression (FIG. 13D). Lastly, IL-1β displayed a trend toward a decrease in Npc1^{-/-}/App^{+/+} (FIG. 13E), which did not reach statistical significance. However, loss of App in a wild-type Npc1 background (Npc1^{+/+}/App^{-/-}) led to a significant decrease in expression (FIG. 5E). Interestingly, loss of App in the NPC brain also tended to decrease IL-1b expression, but such reduction did not reach statistical significance.

### Example 8—Multiplex Protein Analysis Across Npc1 and App Genotypes: NPC Post-Symptomatic Stage

Next, we measured the levels of the 32 prominent pro- and anti-inflammatory cytokines described above in the terminal stage cerebella of mice across the same five genotypes: Npc1^{+/+}/App^{+/+}, Npc1^{-/-}/App^{-/-}, Npc1^{-/-}/App^{+/+}, Npc1^{-/-}/App^{+/-}, and Npc1^{-/-}/App^{-/-}. The average age of the humane endpoint of this animal study were: 11.1 weeks for Npc1^{-/-}/App^{+/+}, 10.4 weeks for Npc1^{-/-}/App^{+/-}, and 9.4 weeks for Npc1^{-/-}/App^{-/-}. Npc1^{+/+}/App^{+/+} and Npc1^{+/+}/App^{-/-} littermates were assessed at 12 weeks of age. In total, 26 cytokines/chemokines were detected in the terminal stage or 12-week cerebella and 20 displayed significant differential expression in either Npc1^{-/-}/App^{+/+}, Npc1^{-/-}/App^{+/-}, or Npc1^{-/-}/App^{-/-} (FIGS. 24A-24N). Levels of six cytokines/chemokines were below the detectable range of the assay (GM-CSF, IL-3, IL-12(p70), IL-13, LIX/CXCL5, and TNFα; data not shown).

For comparative expression analysis, wild-type (Npc1^{+/+}/App^{+/+}) and App gene knockout (Npc1^{+/+}/App^{-/-}) mice were used as primary and secondary controls, respectively. In total, seven cytokines/chemokines were increased in the terminal stage NPC (Npc1^{-/-}/App^{+/+}) cerebella (FIGS. 24A-24G). Of the seven cytokines/chemokines that showed significant increase in Npc1^{-/-}/App^{+/+} mutants compared with wild-type controls (Npc1^{+/+}/App^{+/+}), two (IL-1α and MIP-1β/CCL4) were also increased in Npc1^{-/-}/App^{+/-} and Npc1^{-/-}/App^{-/-} (FIGS. 24A-24B) and two (KC/CXCL5 and LIF) showed a non-significant increase in Npc1^{-/-}/App^{+/-} that reached significance with the loss of both App alleles (Npc1^{-/-}/App^{-/-}) (FIGS. 24C-24D). IP-10/CXCL10 and EOTAXIN/CCL11 showed significant increase in the terminal stage Npc1^{-/-}/App^{+/+} mouse cerebella compared with wild-type controls (Npc1^{+/+}/App^{+/+}), but did not increase in either Npc1^{-/-}/App^{+/-} or Npc1^{-/-}/App^{-/-} samples (FIGS. 24E-24F). RANTES/CCL5 showed significant increase in the terminal stage Npc1^{-/-}/App^{+/+} mouse cerebella compared with wild-type controls (Npc1^{+/+}/App^{+/+}), an effect counteracted by App loss (FIG. 24G).

Two cytokines/chemokines (IL-12(p40) and IL-15) showed significant decrease in the terminal stage Npc1^{-/-}/App^{+/+} mouse cerebella compared with wild-type controls (Npc1^{+/+}/App^{+/+}) and their levels were further decreased in Npc1^{-/-}/App^{+/-} and/or Npc1^{-/-}/App^{-/-} (FIGS. 24H-24I). Lastly, five cytokines/chemokines (IL-5, IL-7, G-CSF, IFN-γ and IL-1β) showed no changes in the terminal stage Npc1^{-/-}/App^{+/+} mouse cerebella compared with wild-type controls (Npc1^{+/+}/App^{+/+}), but their levels were decreased in Npc1^{-/-}/App^{+/-} and/or Npc1^{-/-}/App^{-/-} samples (FIGS. 24J-24N). Altogether, it is interesting to note that cytokine/chemokine expression levels in terminal stage Npc1^{-/-}/App^{+/-} or Npc1^{-/-}/App^{-/-} mouse cerebella were relatively lower than those of Npc1^{-/-}/App^{+/+} (FIGS. 24A-24N). MIP-1P/CCL4 was the only exception to this general pattern (FIG. 24B).

### Example 9—T Cell Infiltration Across Npc1 and App Genotypes

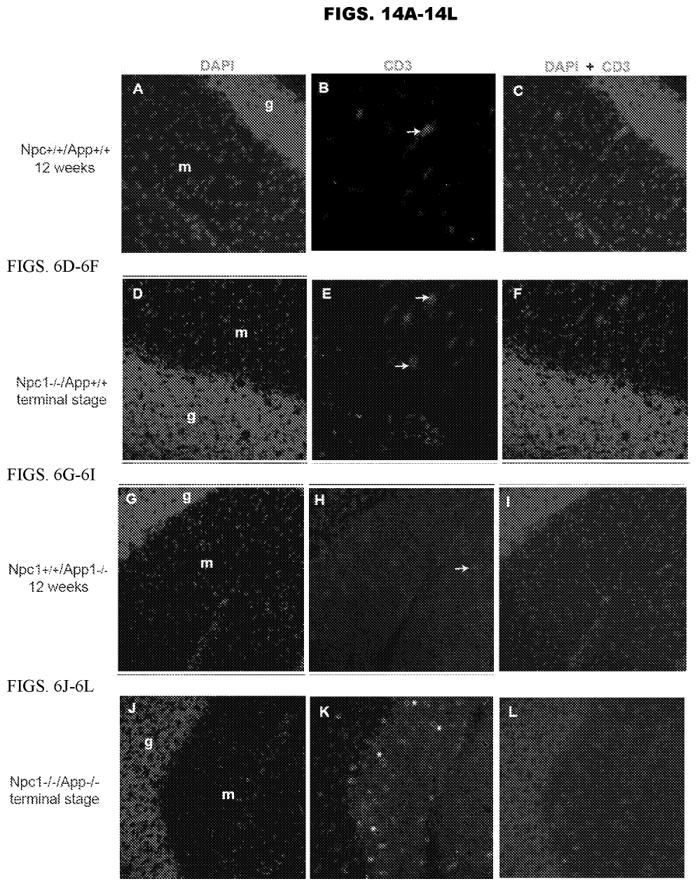

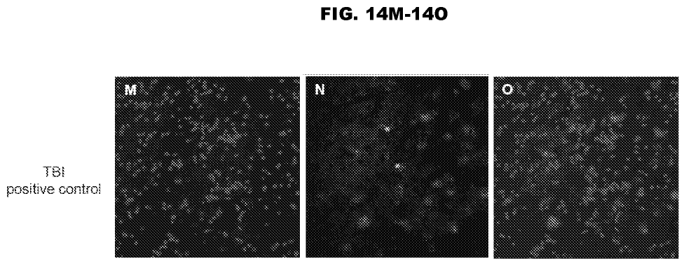

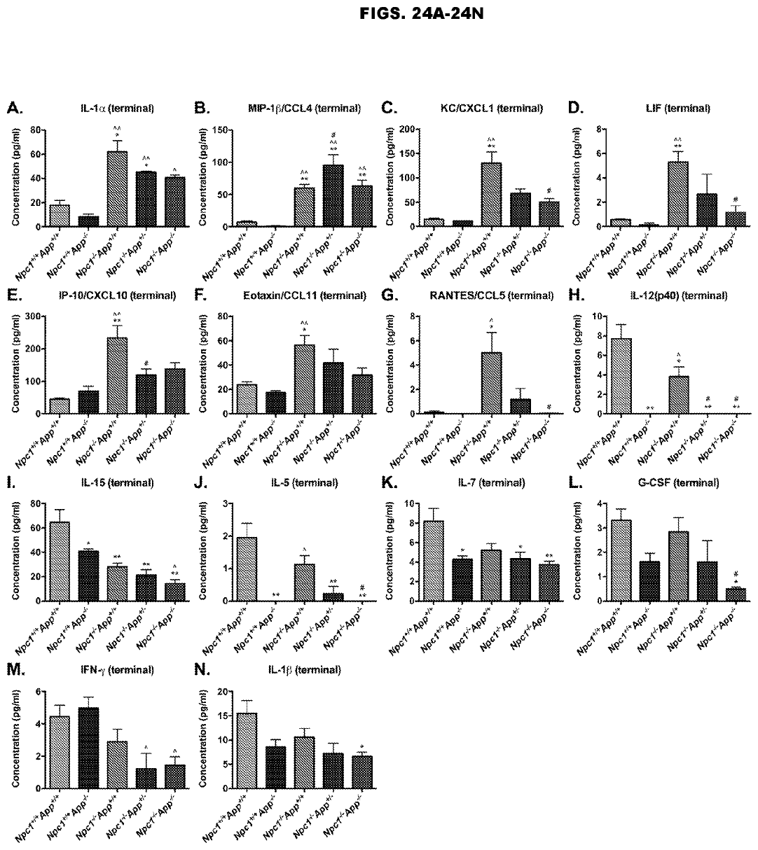

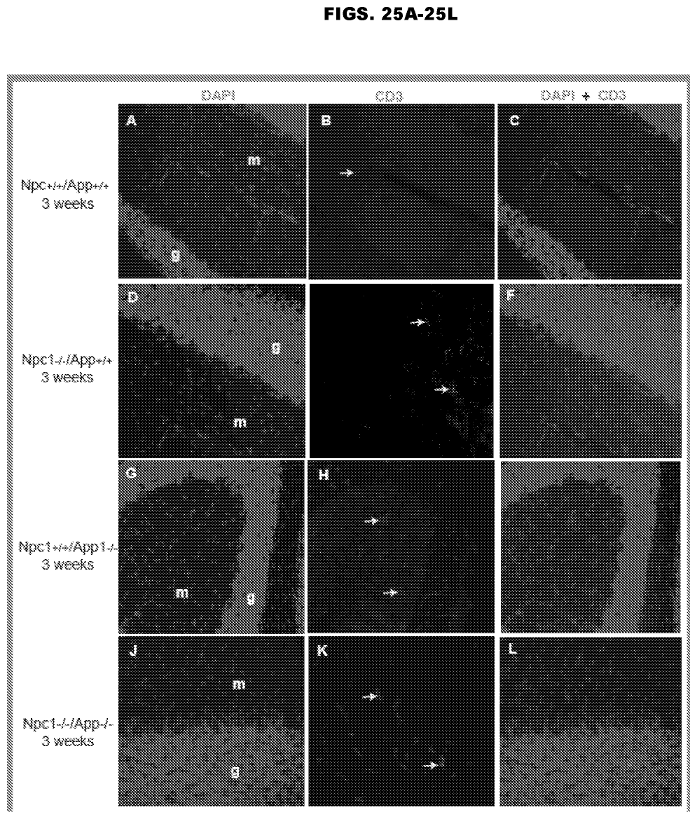

Because of the effect of IP-10 increased expression on T cell activation and chemotaxis, T cell infiltration was measured across Npc1^{+/+}/App^{+/+}, Npc1^{+/+}/App^{-/-}, Npc1^{-/-}/App^{+/+} and Npc1^{-/-}/App^{-/-} genotypes, at three weeks of age as well as 12 weeks (Npc1^{+/+}/App^{+/+}, Npc1^{+/+}/App^{-/-}) or humane endpoint terminal stage (Npc1^{-/-}/App^{+/+} and Npc1^{-/-}/App^{-/-}). As shown in FIGS. 14A-14O, T cell infiltration was indeed evident in Npc1^{-/-}/App^{-/-} cerebellum at terminal stage (FIGS. 14J-14L). FIGS. 14A-14O are immunohistochemically stained-images of T cells in cerebellum. Shown for comparison as a positive control is CD3 staining of T cells in mice following a traumatic brain injury protocol: FIGS. 14A-14C—Npc1^{+/+}/App^{+/+} mice at 12 weeks of age, FIGS. 14D-14F—Npc1^{-/-}/App^{+/+} mice at terminal disease stage, FIGS. 14G-14I—Npc1^{+/+}/App^{-/-} mice at 12 weeks of age, FIGS. 14J-14L—App^{-/-}/Npc1^{-/-} mice at terminal disease stage, FIGS. 14M-14O—Traumatic brain injury positive control. Shown is the lesion area. g: granular layer of the cerebellum; in: molecular layer of the cerebellum. White asterisks show CD3+ cells and white arrows show areas of stained patterns that are artifacts, as they appear in all genotypes and all ages tested. No evidence of T cell infiltration was found in 3-week old mice of any Npc1 or App genotypes (FIGS. 25A-25L). FIG. 25A-25L are immunohistochemically stained-images to examine the infiltration of CD3+ T cells in cerebellum. Immunohistochemically staining reveals the absence of CD3+ cells in the cerebellum of mice of wild type, Npc1^{-/-}, App^{-/-} and App^{-/-}/Npc1^{-/-} mice at 3 weeks of age. FIGS. 25A-25C are images of Npc1^{+/+}/App^{+/+} mice cerebella. FIGS. 25D-25F are images of Npc1^{-/-}/App^{+/+} mice cerebella. FIGS. 25G-25I are images of Npc1^{+/+}/App^{-/-} mice cerebella. FIGS. 25J-25L are images of App^{-/-}/Npc1^{-/-} mice cerebella. g: granular layer of the cerebellum; m: molecular layer of the cerebellum. White arrows show areas of stained patterns that are artifacts, as they appear in all genotypes and all ages tested.

The comparative and systematic genome-wide transcriptome analyses of Npc1^{+/+}/App^{+/+}, Npc1^{+/+}/App^{-/-}, Npc1^{-/-}/App^{+/+}, and Npc1^{-/-}/App^{-/-} mice at pre-symptomatic stage revealed that loss of APP function results in severe exacerbation of multiple inflammatory pathways already present in the NPC brain. Specifically, GSEA and IPA Upstream Analysis showed significantly increased expression of IFN-γ- and IFN-α-responsive genes in the Npc1^{-/-}/App^{-/-} cerebellar transcriptome (FIGS. 9-12; 262 IFN-γ-responsive and 84 IFN-α-responsive genes; FIGS. 10 and 12), when compared with Npc1^{-/-}/App^{+/+} mice (60 IFN-γ-responsive and 23 IFN-α-responsive genes, consistent with the significant exacerbation of all four major inflammatory pathways previously identified in this mouse model of NPC, namely activation of microglia, anti-viral response, activation of T-lymphocytes, and chemotaxis of T-lymphocytes (FIGS. 15, 16, 18, and 20). The mechanisms by which APP loss may cause an exacerbation of inflammatory pathways prior to disease onset in NPC is not immediately clear. APP processing is abnormal in the NPC brain, as evidenced by an increase in amyloid peptide AP expression, possibly due to the formation of aberrantly enlarged endosomes, a necessary compartment for the generation of A. Thus, it would appear reasonable to link excess Δβ expression in the NPC with its pathogenesis. However, loss of APP and, by extension, of Δβ, in the NPC brain, leads to decreased life span, increased cholesterol abnormalities and, notably, disruption of tau homeostasis, as well as an early exacerbation of inflammation, as shown here. These findings suggest that ΔP expression is not a primary pathogenic factor in NPC. Rather, given that APP is a multi-potent cytoprotective molecule, whose cleaved products provide beneficial effects against oxidative stress, metabolic stress, and pathogenic infections, it seems more likely that APP plays a homeostatic role in the brain and that loss of that role accelerates NPC onset and progression. For example, both monomeric and oligomeric forms of ΔP have been characterized to possess potent anti-oxidant activity and the function of APP intracellular domain (AICD) as a transcription factor has recently been shown to directly regulate the cytoprotective mechanisms against oxysterol-mediated stress. Furthermore, ΔP has potent anti-microbial activity against many strains of pathogens, including bacteria, viruses, and yeast.

Overall, the available evidence suggests that loss of APP function in the Npc1^{-/-}/App^{-/-} brain contributes to the early altered expression of genes directly related to immune response pathways against pathogens, including Antimicrobial Response and Antiviral Response identified by IPA analysis (FIGS. 16 and 17). Interestingly, compared with the sole activation of Antiviral Response identified by IPA in pre-symptomatic NPC, APP loss resulted in an additional enrichment of the larger functional Antimicrobial Response, which included 31 additional antimicrobial genes (FIG. 16). This increase in anti-microbial function is further highlighted by the activation of genes involved in T-lymphocyte activation and chemotaxis, as well as the activation of antigen presenting cells, all of which are crucial in host-immune response against various strains of pathogens (FIGS. 18-21).

It is also noteworthy that changes in gene expression in pre-symptomatic NPC as a result of App deletion (Npc1^{-/-}/App^{-/-}) translated into increased expression of pro-inflammatory cytokines and chemokines (FIGS. 13A-13E), even with the loss of one single App allele. This was the case with the protein expression of IP-10/CXCL10, the central downstream effector of IFN-γ identified in pre-symptomatic Npc1^{-/-} mice (FIG. 13A), as well as several other cytokines, including RANTES, eotaxin/CCL11 and IL-10 (FIGS. 13B-13D). FIG. 13E is a graphical representation of the expression of IL-1p expression in Npc1^{-/-}/App^{+/-} and/or Npc1^{-/-}/App^{-/-} mouse cerebella compared with wild-type (Npc1^{+/+}/App^{+/+}) and/or Npc1^{-/-} /App^{-/-}. Interestingly, the notion that haploinsufficiency of APP is a risk factor for neurotoxicity has been proposed in a model of copper-mediated CNS cytotoxicity. In that study, a single allele loss of App in mice was sufficient to alter copper homeostasis comparable to that of mice lacking both alleles of App. Therefore, it is plausible that dysregulation of APP function may exacerbate the inflammatory response and poor prognosis of NPC in humans.

Functionally, IP-10/CXCL10 is a potent downstream effector of IFN-γ, the master regulator of the adaptive immune activation that is crucial in the transition from the innate immune response to the antigen-specific adaptive immune response. IP-10/CXCL10 binds to CXCR3, on activated immune cells such as activated T-lymphocytes or natural killer cells to drive the chemotaxis of CXCR3+ cells to the site of inflammation. Furthermore, IP-10/CXCL10 also plays a major role in the development and antigen-specific activation of T-lymphocytes. In addition, interferon-inducible T-cell alpha chemoattractant (I-TAC/CXCL11) also binds the same CXCR3 receptor to elicit similar physiological functions. The fact that T cell infiltration is apparent in the Npc1^{-/-}/App^{-/-} cerebellum (FIGS. 14A-14O) supports the notion that APP loss may exert its deleterious effect through IP-10/CXCL10-driven T lymphocyte activation and chemotaxis.

In both Npc1^{-/-}/App^{+/-} and Npc1^{-/-}/App^{-/-} mouse cerebella, another major cytokine significantly increased at 3 weeks of age was eotaxin/CCL11 (FIG. 13C). Eotaxin/CCL11 is a potent eosinophil chemoattractant, implicated in various eosinophil-related pathogenic processes such as asthma and airway inflammation. While the combined functional roles of eosinophils and eotaxin/CCL11 are widely characterized in the periphery, the precise role of both in the CNS is not well defined. For example, eotaxin/CCL11 is an anti-inflammatory Th2 cytokine in the CNS in a murine model of multiple sclerosis. On the other hand, astrocyte-mediated release of eotaxin/CCL11 and subsequent enhancement of neuronal death via increased production of microglial reactive oxygen species have also been reported. In the context of the early and widespread activation of IFN-γ-responsive signaling that occurs in pre-symptomatic NPC brains, IFN-γ potentiates the subsequent release of eotaxin/CCL11 in the periphery, thereby suggesting a potential for the co-activation of IFN-γ and eotaxin/CCL11 under certain inflammatory conditions. Interestingly, co-expression of IP-10/CXCL10 receptor CXCR3 and eotaxin/CCL11 receptor CCR5 (whose ligands also include MIP-1α/CCL3, MIP-1β/CCL4, and RANTES/CCL5) have been characterized in autoimmune T-lymphocytes, consistent with the co-activation of CXCR3 and CCR5 as a potential pathologic mechanism involved in autoimmunity.

Loss of APP also showed a significant impact on the expression pattern of cytokines and chemokines in terminal-stage brains, as illustrated in FIGS. 24A-24N. Interestingly, the overall expression of pro-inflammatory cytokines and chemokines in the terminal stage Npc1^{-/-}/App^{+/-} or Npc1^{-/-}/App^{-/-} were relatively lower than that of Npc1^{-/-}/App^{+/+} (FIGS. 24A-24N). While the precise mechanism responsible for this phenomenon remains to be elucidated, one plausible explanation is the significant reduction in brain mass and paralleled neuronal death observed in the Npc1^{-/-}/App^{-/-} terminal stage cerebella. Contrary to the classical understanding of neuronal secretion of cytokines, recent evidence consistently highlights neurons as a major source of proinflammatory cytokines and chemokines under various cytotoxic stresses within the CNS. The difference in age-at-collection may be another confounding factor for the terminal stage cytokine/chemokine expressions, as the average age for humane-endpoint varied by a week with the successive loss of an App allele (11.1 weeks for Npc1^{-/-}/App^{+/+}, 10.4 weeks for Npc1^{-/-}/App^{+/-}, and 9.4 weeks for Npc1^{-/-}/App^{-/-}, Npc1^{+/+}/App^{+/+}).

Loss of APP function in the NPC brain exacerbates the pathogenic neuroinflammation that occurs prior to symptomatic onset, exerting a direct impact on the four major inflammatory pathways previously identified in this mouse model of NPC, namely activation of microglia, anti-viral response, activation of T-lymphocytes, and chemotaxis of T-lymphocytes. These findings shed a new light into the function of APP as a cytoprotective modulator in the CNS, offering potential much-needed evidence-based therapies against NPC.

What is claimed is:

1. A method of treating Niemann-Pick disease type C in a subject, comprising administering to the subject an immunomodulator.

2. The method of claim 1, wherein the immunomodulator is an immunosuppressor.

3. The method of claim 2, wherein the immunosuppressor is an inhibitor of Interferon I.

4. The method of claim 2, wherein the immunosuppressor is an inhibitor of Interferon II.

5. The method of claim 2, wherein the immunosuppressor is an inhibitor of interferon-gamma induced protein 10.

6. The method of claim 2, wherein the immunosuppressor is an inhibitor of a toll-like receptor.

7. The method of claim 2, wherein the immunosuppressor is an inhibitor of T-cell function.

8. The method of claim 1, wherein the immunomodulator is Neuregulin 1.

9. The method of claim 1, wherein the immunomodulator is an inhibitor of a fatty acid binding protein.

10. The method of claim 1, wherein the immunomodulator is fingolimod.

11. A method of treating Niemann-Pick disease type C in a subject, comprising administering to the subject a modulator of amyloid precursor protein (APP) function.

12. The method of claim 11, wherein the modulator of APP function is a serotonin receptor agonist.

13. The method of claim 12, wherein the serotonin receptor agonist is donecopride.

14. The method of claim 11, wherein the modulator of APP function is a specific 5-HT4 receptor agonist.

15. A method of treating Niemann-Pick disease type C (NPC) in a subject, comprising administering to the subject a combination of an immunomodulator and a modulator of amyloid precursor protein (APP) function.

16. The method of claim 15, wherein the immunomodulator is one or more therapies selected from:
(a) at least one interferon (IFN) inhibitor;
(b) at least one IP10/CXCL10 inhibitor;
(c) at least one CXCR3 inhibitor;
(d) at least one inhibitor of MIG/CXCL9, RANTES/CCL5, EOTAXIN/CCL11, and IL-10;
(e) at least one inhibitor of TLR; and
(f) at least one inhibitor of MCP1/CCL2, MIP-1α/CCL3, MIP-1β/CCL4, IL-1α, and KC/CXCL1.

17. The method of claim 15, wherein the modulator of APP function is a serotonin receptor agonist.

18. The method of claim 17, wherein the serotonin receptor agonist is donecopride.

19. The method of claim 15, wherein the modulator of APP function is a specific 5-HT4 receptor agonist.

20. A method of delaying onset of Niemann-Pick disease type C (NPC) in a subject, comprising administering to the subject an immunomodulator, or a modulator of amyloid precursor protein (APP) function, or a combination thereof.
